(19) United States
(12) Patent Application Publication
(10) Pub. No.: US 2009/0281114 A1
(43) Pub. Date: Nov. 12, 2009

(54) INDOLYLALKYL DERIVATIVES OF PYRIMIDINYLPIPERAZINE AND METABOLITES THEREOF FOR TREATMENT OF ANXIETY, DEPRESSION, AND SEXUAL DYSFUNCTION

(75) Inventors: Stephen J. KRAMER, Houston, TX (US); Louis F. FABRE, Houston, TX (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: Fabre-Kramer Pharmaceuticals, Inc., Houston, TX (US)

(21) Appl. No.: 12/437,220

(22) Filed: May 7, 2009

Related U.S. Application Data
(60) Provisional application No. 61/051,571, filed on May 8, 2008.

(51) Int. Cl. A61K 31/506; A61P 3/04; A61P 25/28; A61P 25/22; A61P 25/32; A61P 25/30; A61P 15/00

(52) U.S. Cl. 514/252.19

(57) ABSTRACT

The present invention relates to a method for alleviation, prevention, and treatment of anxiety, depression, and sexual dysfunction by administering certain indolylalkyl derivatives of pyrimidinylpiperazine or metabolites thereof. A preferred embodiment is of the following formula:

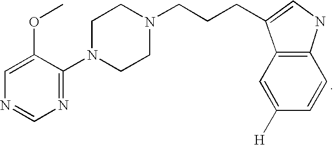

### BACKGROUND OF THE INVENTION

1. Field of the Invention

The present invention relates to a method for alleviation, prevention, and treatment of anxiety, depression, and sexual dysfunction by administering certain indolylalkyl derivatives of pyrimidinylpiperazine or metabolites thereof. A preferred embodiment is of the following formula:

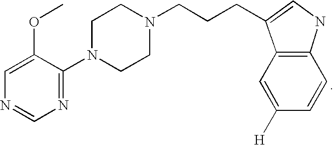

2. Discussion of the Background

This invention generally pertains to heterocyclic carbon compounds having drug and bio-affecting properties and to their preparation and use. In particular the invention is concerned with 1,4-disubstituted piperazine derivatives wherein one substituent moiety is an indol-3-yl-alkyl group and the other moiety is an alkoxy-substituted pyrimidin-4-yl ring. These compounds possess a unique serotonergic profile that renders them useful in treatment of anxiety, depression, and sexual dysfunction.

Archer disclosed a large series of CNS-depressant indolylalkylpiperazines in U.S. Pat. No. 3,188,313. Among a large number of possible substituents on the 4-nitrogen atom of the piperazine ring was pyrimidine (unsubstituted). In U.S. Pat. No. 3,562,278, Archer disclosed and claimed a series of 1-indolylethyl-4-substituted-piperazines. Among the possible 4-substituents listed is 2-pyrimidinyl, again unsubstituted.

Dowie, et al. disclosed a series of 3-alkylaminoindole derivatives as being potentially useful for the treatment of migraine in a published patent application, GB 2,124,210. One member of this series of compounds was specifically claimed in a later patent application of Oxford, GB 2,162,522, published Feb. 5, 1986. This particular compound is known in the literature as sumatriptan.(1)

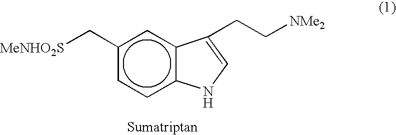

A series of novel indoline derivatives were disclosed Feb. 7, 1990 by Manoury, et al., in European patent application EPA 354,094. These compounds are described as being useful for treatment of various CNS disorders including depression, anxiety and migraine. Included among these art compounds are those of formula (2)

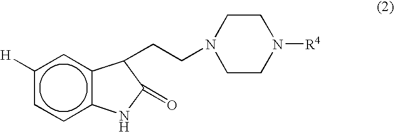

wherein R⁴ is aryl, pyridine or quinoline moieties.

U.S. Pat. No. 5,300,506 discloses a series of novel indol-3-ylalkyl derivatives of alkoxypyrimidinylpiperazines intended to be useful agents for alleviation of vascular headache on the basis of their potent affinity and agonist activity at 5-HT_{1D} binding sites. None of these art compounds make obvious the instant novel indol-3-ylalkyl derivatives of alkoxypyrimidinylpiperazine for anxiety, depression, and sexual dysfunction.

Anxiety

Anxiety is a condition characterized by cognitive, physical, emotional, and behavioral components, combining to result in feelings typically recognized as fear, apprehension, or worry. Anxiety is often accompanied by physical sensations such as heart palpitations, nausea, chest pain, shortness of breath, stomach aches, or headache. The cognitive component entails recognition of a threat of certain danger. Physically, the body prepares the organism to deal with such a threat, including increased blood pressure, heart rate, perspiration, and blood circulation to major muscle groups. The body also experiences inhibition of the immune and digestive system functions. External signs of anxiety may include pale skin, sweating, trembling, or pupillary dilation. Emotionally, anxiety causes a sense of dread or panic and physically causes nausea, diarrhea, and chills. Behaviorally, both voluntary and involuntary behaviors may arise directed at escaping or avoiding the source of anxiety. Anxiety is not always pathological. Rather, it is a common emotion along with fear, anger, sadness, and happiness, with an important function in relation to survival. However, anxiety can be maladaptive, with the most extreme cases being expressed as anxiety disorders.

Anxiety can be classified by its severity. Severe anxiety disorders only affects a small minority of anxiety sufferers, and may include severe panic and anxiety disorders like obsessive-compulsive disorder (OCD), post-traumatic stress disorder (PTSD), social phobias, and stress disorders. Severe anxiety disorders are highly treatable but require medical diagnosis. Generalized anxiety disorder, characterized as by mild to moderate anxiety, is far more common but harder to identify. Generalized anxiety disorder is characterized by compulsive worrying and physical symptoms of anxiety which persist for more than six months.

The causes of anxiety are not completely understood. It is likely caused by an interrelationship between multiple factors. It is known that a neurotransmitter imbalance can sensitize a person's brain to experiencing anxious feelings or fear. Consistently high levels of excitatory neurotransmitters, such as norepinephrine and epinephrine, along with low levels of inhibitory neurotransmitters, such as serotonin and gamma-aminobutyric acid, may result in predisposition to anxiety disorders. Caffeine is known to result in increased levels of dopamine, and sufficient amounts may result in anxiety. The neurotransmitter imbalances that cause anxiety are related to those in children with ADHD and ADD, which are associated with higher levels of dopamine.

In addition to neurotransmitter imbalances, imbalances in the hypothalamic-pituitary-adrenal (HPA) axis can influence anxiety. The hypothalamus releases corticotropin-releasing factor (CRF) as part of the cascade of hormones in the fight-or-flight response. CRF stimulates adrenocorticotropic hormone (ACTH), which in turn acts on the adrenal glands, which release cortisol. In normal situations, cortisol allows for the rush of glucose, fat, and protein that gives cells increased energy and alertness. In healthy individuals, the hormonal release recedes once the perceived threat is addressed. Disruption of the HPA axis, however, can result in persistently increased levels of ACTH and cortisol, resulting in anxiety. Other hormones, including sex hormones, may affect anxiety. For example, higher levels of estrogen, along with lower levels of progesterone, may result in decreased serotonin levels.

Although acute attacks of anxiety are not experienced by every anxiety sufferer, they are a common symptom. Anxiety attacks usually come without warning, and although the fear is generally irrational, the perceived danger is very real. A person experiencing an anxiety attack will often feel as if they are about to die or pass out, may experience fear, and may feel the need to avoid certain stressful situations or social situations due to fear of embarrassment. The person may experience considerable confusion and irritability when experiencing anxiety. Physical symptoms include hot flushes, chest pain, sudden exhaustion, headaches, and shortness of breath, digestive problems, and nausea.

Anxiety may be treated with medication, therapy, or a combination of both. Other treatment modalities may include self-help techniques, lifestyle changes, and alternative medicine for relief and wellness. Medications used for treatment of anxiety include antidepressants. These may include selective serotonin reuptake inhibitors, tricyclic antidepressants, and monoamine oxidase inhibitors. Other antianxiety drugs include benzodiazepines and buspirone. While these may be effective, the side effects of such treatments can be significant, including the development of dependency or addiction. The frequency of side effects, potential addictiveness, and limited success of stimulant drugs has led to a search for alternate means of treating or preventing anxiety.

Depression

Depression is a psychiatric disorder, characterized by a pervasive low mood, loss of interest in usual activities and diminished ability to experience pleasure. Clinical depression affects about 8-17% of the population on at least one occasion in their lives, before the age of forty. In the United States, it has been estimated that approximately 14 million adults suffer depression per year. Although the term “depression” is commonly used to describe a temporary condition when one “feels blue”, clinical depression is a serious and often disabling condition that can significantly affect a person's work, family and school life, sleeping and eating habits, general health and ability to enjoy life. The course of clinical depression varies widely. Depression can be a once in a life-time event or have multiple recurrences. It can appear either gradually or suddenly, and either last for a few months or be a life-long disorder. Depression is a major risk factor for suicide, and people with depression suffer from higher mortality due to other causes of death.

Clinical depression may be a primary condition, or it may be secondary to another condition, such as chronic pain. Clinical depression can present with a variety of symptoms. Most patients display a marked change in mood, a deep feeling of sadness, and a noticeable loss of interest or pleasure in favorite activities. Other symptoms include persistent sadness or anxiety, loss of appetite and/or weight loss, increased appetite and overeating and weight gain, insomnia, restlessness or irritability. The depressed patient may experience feelings of worthlessness, inappropriate guilt, helplessness, hopelessness, and/or pessimism. He may have difficulty thinking, concentrating, remembering or making decisions. The depressed patient may have suicidal thoughts, and may attempt suicide. Decreased energy, fatigue, and sluggishness, along with persistent physical symptoms that do not respond to treatment, such as headaches, digestive problems, and chronic pain, are often experienced by depressed patients. Diagnosis of depression is detailed in Diagnostic and Statistical Manual of Mental Disorders (Fourth Ed.) (American Psychiatric Association (1994), herein incorporated by reference in its entirety.

There are numerous theories about causes of depression. It is generally believed that clinical depression is caused by an influence of some combination of biological and genetic factors, environmental influences, and childhood or developmental events. Several risk factors have been identified. In the United States, women are about as twice as likely as men to be diagnosed and treated for major depression. People over the age of 65 may be especially vulnerable. Previous episodes increase the risk of depression, as approximately one-half of those who have developed depression will experience it again. And people who have relatives who have had clinical depression have a greater chance of developing it themselves, suggesting a genetic component.

The biological causes of clinical depression may include disordered brain function, neurotransmitters, and hormones. Researchers studying clinical depression tend to look at several aspects of brain function including the structures of the limbic system and the function of neurotransmitters within neurons. Researchers have identified associations between clinical depression and the function of serotonin, norepinephrine, and dopamine. These three neurotransmitters function within structures of the brain that regulate emotions, reactions to stress, and the physical drives of sleep, appetite, and sexuality.

Neurotransmitters play a role in the development or treatment of clinical depression. It has been shown that many people who are depressed have low levels of the neurotransmitter norepinephrine. The use of some antidepressants can increase the level of norepinephrine in the brain, and subsequently relieve depressive symptoms. However, some depressed people have high levels of norepinephrine, making the causation link unclear.

The dopamine system is an important factor in depression. Short-term surges of dopamine are normally associated with feelings of pleasure. However, abnormally high concentrations of the chemical messenger may be an important cause of depression.

The endocrine system is also involved in the causation of clinical depression. The hormones that are released into the body by the endocrine glands regulate processes such as reaction to stress and sexual development. A great number of depressed people have abnormal levels of some hormones in their blood despite having healthy glands, which is believed to result in problems with appetite and sleeping. Problems with hormone levels may be intertwined with the changes in brain chemistry that are seen in clinical depression. The endocrine system is connected with the brain at the hypothalamus which controls many bodily activities such as sleep, appetite, and sexual drive. The hypothalamus also regulates the pituitary gland that, in turn, controls the hormonal secretion of other glands. The hypothalamus uses some of the neurotransmitters that have been associated with depression as it manages the endocrine system. These neurotransmitters, serotonin, norepinephrine, and dopamine all have a role in the management of hormone function.

Genetic factors and environmental factors, such as prolonged stress at home or work, coping with the loss of a loved one, or traumatic events, may also contribute to depression.

Sexual Dysfunction

Sexual Dysfunction may be defined as difficulty during any stage of the sexual act (which includes desire, arousal, orgasm, and resolution) that prevents the individual or couple from enjoying sexual activity. Sexual dysfunction disorders are generally classified into four categories: sexual desire disorders, sexual arousal disorders, orgasm disorders, and sexual pain disorders.

Sexual desire disorders (decreased libido) may be caused by a decrease in the normal production of estrogen (in women) or testosterone (in both men and women). Other causes may be aging, fatigue, pregnancy, and medications (such as anti-depressants such as fluoxetine, sertraline, and paroxetine are well known for reducing desire in both men and women. Psychiatric conditions, such as depression and anxiety, can also cause decreased libido.

Sexual arousal disorders were previously known as frigidity in women and impotence in men. These have now been replaced with less judgmental terms. Impotence is now known as erectile dysfunction, and frigidity is now described as any of several specific problems with desire, arousal, or anxiety. For both men and women, these conditions may appear as an aversion to, and avoidance of, sexual contact with a partner. In men, there may be partial or complete failure to attain or maintain an erection, or a lack of sexual excitement and pleasure in sexual activity.

Orgasm disorders are a persistent delay or absence of orgasm following a normal sexual excitement phase. The disorder occurs in both women and men. Again, the SSRI antidepressants are frequent culprits—these may delay the achievement of orgasm or eliminate it entirely.

Sexual pain disorders affect women almost exclusively, and are known as dyspareunia (painful intercourse) and vaginismus (an involuntary spasm of the muscles of the vaginal wall, which interferes with intercourse). Dyspareunia may be caused by insufficient lubrication (vaginal dryness) in women.

Sexual dysfunctions are more common in the early adult years, with the majority of people seeking care for such conditions during their late 20s through 30s. The incidence increases again in the geriatric population, typically with gradual onset of symptoms that are associated most commonly with medical causes of sexual dysfunction. Sexual dysfunction is more common in people who abuse alcohol and drugs. It is also more likely in people suffering from diabetes and degenerative neurological disorders. Ongoing psychological problems, difficulty maintaining relationships, or chronic disharmony with the current sexual partner may also interfere with sexual function.

Symptoms of sexual dysfunction may include loss of libido, inability to feel aroused, painful intercourse in both male and female patients. In men, symptoms may include inability to attain or maintain an erection, delay or absence of ejaculation, and inability to control timing of ejaculation. In women, symptoms may include inability to relax vaginal muscles enough to allow intercourse, inadequate vaginal lubrication before and during intercourse, inability to attain orgasm, and burning pain on the vulva or in the vagina with contact to those areas.

Sexual dysfunction is common among individuals with depression. Depressed individuals show decreased sexual interest and reported reduced levels of arousal. Sexual dysfunction is also a common side effect of antidepressant treatment, particularly pharmacotherapy with serotonin reuptake inhibitors (SRIs). The sexual response cycle consists of 4 phases: desire, arousal, orgasm, and resolution. All of these phases may be affected by reproductive hormones and neurotransmitters. Estrogen, testosterone, and progesterone promote sexual desire. Dopamine promotes desire and arousal. Norepinephrine promotes arousal. Prolactin inhibits arousal. Oxytocin promotes orgasm. Serotonin may have a negative impact on the desire and arousal phases of the sexual response cycle, possibly due to its inhibition of dopamine and norepinephrine.

Treatment of sexual dysfunction involves identifying the specific cause and, often, treating the underlying condition. Medical causes that are reversible or treatable are usually managed medically or surgically. Physical therapy and mechanical aides may prove helpful for some people experiencing sexual dysfunction due to physical illnesses, conditions, or disabilities.

Neurological and psychological factors play an important role in sexual dysfunction. Anxiety, fear, and depression can particularly be addressed with treatments such as psychological therapy and medications. Dopamine is known to promote desire and arousal. Accordingly, dopaminergic agents may be helpful for the treatment of antidepressant-induced sexual dysfunction.

Therapy for the treatment of ADD, anxiety, depression, and/or sexual dysfunction may be to target the serotonin system. Deregulation of this system has been linked with each of these conditions. For example, buspirone exhibits an affinity for a series of receptors including serotonin receptors, dopamine receptors, and α-adrenergic receptors. The serotonergic activity may be related to improved behavior and impulsivity. However, current therapies have not been uniformly successful.

Accordingly, there remains a critical need for novel treatment strategies of patients suffering from anxiety, depression, and sexual dysfunction. Moreover, there remains a critical need for treatment strategies, which are safe and effective with a reduction in or elimination of any side effects associated with existing treatment strategies. Thus, the objectives of the present invention relate to the use of novel indol-3-ylalkyl derivatives of alkoxypyrimidinylpiperazines and their derivatives to provide treatment of anxiety, depression, and sexual dysfunction.

### SUMMARY OF THE INVENTION

The method of the present invention is intended for the alleviation of anxiety, depression, and sexual dysfunction. The method essentially involves administration of an indol-3-ylalkyl derivative of alkoxypyrimidinylpiperazines, or a pharmaceutically acceptable salt and/or solvate thereof, to a human in need of such treatment. For use in the instant method, oral and transnasal administration of pharmaceutical compositions containing the subject agents are preferred.

In a broad aspect, the present invention is concerned with treating anxiety, depression, and sexual dysfunction with indol-3-ylalkyl derivatives of alkoxypyrimidinylpiperazines. A specific aspect concerns novel compounds having useful serotonergic properties and characterized by Formula I

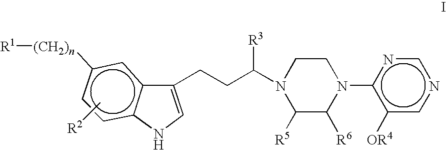

In Formula I, R¹ is a substituent selected from a group of substituents comprising hydrogen; halogen lower alkyl; lower alkoxy; phenyl-lower alkoxy, with the phenyl ring unsubstituted or bearing a lower alkyl substituent; amino; cyano; hydroxy; nitro; —OCH₂CN; —OCH₂CONR⁷R⁸; —SO₂NR⁷R⁸; —O₂CR⁹; —SO₂R⁹; —O₂CNR⁷R⁸; —COR⁸; —CO₂R⁹; —CONR⁷R⁸; —NR⁷CO₂R⁹; —NR⁷COR⁸; —NR⁷SO₂R⁹; and

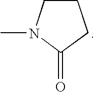

R² is selected from hydrogen, halogen, lower alkyl, lower alkoxy and —CO₂R⁹.
R³, R⁵, R⁶ and R⁷ are independently selected from hydrogen and lower alkyl. R³ however cannot be hydrogen when the R¹—(CH₂)ₙ moiety is hydrogen, halogen, lower alkyl, lower alkoxy or —CONH₂ and R² is hydrogen, halogen or lower alkyl.
R⁴ is lower alkyl.
R⁸ is selected from hydrogen, lower alkyl, R⁷-substituted phenyl-lower alkyl and trifluoromethyl.
R⁹ is selected from lower alkyl and R⁷-substituted phenyl-lower alkyl.
The symbol n is zero or the integers 1 or 2.

Additionally compounds of Formula I also encompass all pharmaceutically acceptable acid addition salts and/or solvates thereof. Hydrates are the preferred solvates. The present invention is also considered to include stereoisomers as well as optical isomers e.g. mixtures of enantiomers as well as individual enantiomers and diasteromers, which arise as a consequence of structural asymmetry in certain compounds of the instant series. Separation of the individual isomers is accomplished by application of various methods which are well known to practitioners in the art. The term “lower alkyl” refers to both straight and branched chain carbon radicals of from 1 to 4 carbon atoms inclusive. Illustrative of these radicals are carbon chains which can be methyl, ethyl, propyl, isopropyl, 1-butyl, 1-methylpropyl 2-methylpropyl.

Preferred compounds of Formula I are those wherein R¹ is a polar substituent attached to the 5-position of the indole ring.

The pharmaceutically acceptable acid addition salts of the invention are those in which the counter-ion does not contribute significantly to the toxicity or pharmacological activity of the salt and, as such, they are the pharmacological equivalents of the bases of Formula I. They are generally preferred for medical usage. In some instances, they have physical properties which makes them more desirable for pharmaceutical formulation such as solubility, lack of hygroscopicity, compressibility with respect to tablet formation and compatibility with other ingredients with which the substance may be used for pharmaceutical purposes. The salts are routinely made by admixture of a Formula I base with the selected acid preferably by contact in solution employing an excess of commonly used inert solvents such as water, ether, benzene, methanol, ethanol, ethyl acetate and acetonitrile. They may also be made by metathesis or treatment with an ion exchange resin under conditions in which the anion of one salt of the substance of the Formula I is replaced by another anion under conditions which allow for separation of the desired species such as by precipitation from solution or extraction into a solvent, or elution from or retention on an ion exchange resin. Pharmaceutically acceptable acids for the purposes of salt formation of the substances of Formula I include sulfuric, phosphoric, hydrochloric, hydrobromic, hydroiodic, citric, acetic, benzoic, cinnamic, fumaric, mandelic, oxalic, phosphoric, nitric, mucic, isethionic, palmitic, heptanoic, and others.

The compounds of Formula I can be prepared by adaptation of the general process shown in Scheme 1. Processes for syntheses of intermediate compounds are outlined in Scheme 2. In addition, certain compounds and their syntheses will be set forth in more detail in the Specific Embodiments section, infra.

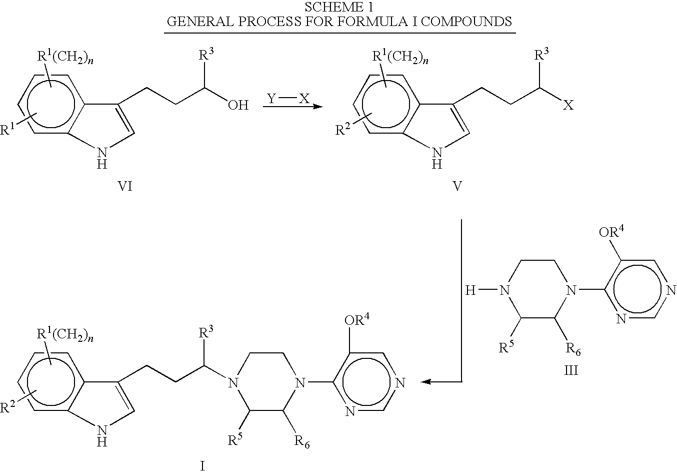

In the process of Scheme 1, R¹ through R⁶ and n are as defined supra. The reagent Y—X represents an organic leaving group reagent wherein X is the leaving group fragment such as tosyl, mesyl, halide, sulfate, phosphate and so forth; and Y is either a proton or a counter ion: e.g. Y—X can be HBr, mesyl or tosyl chloride and the like. The reactions of Scheme I and their application are familiar to the practitioner skilled in organic synthesis and modifications of conditions and reagents would be readily understood.

An indolylalkyl alcohol of formula VI is converted to an activated intermediate of formula V in which the alcoholic moiety is converted into an organic leaving group. Reaction of intermediate V with a pyrimidinylpiperazine of formula III then provides product I.

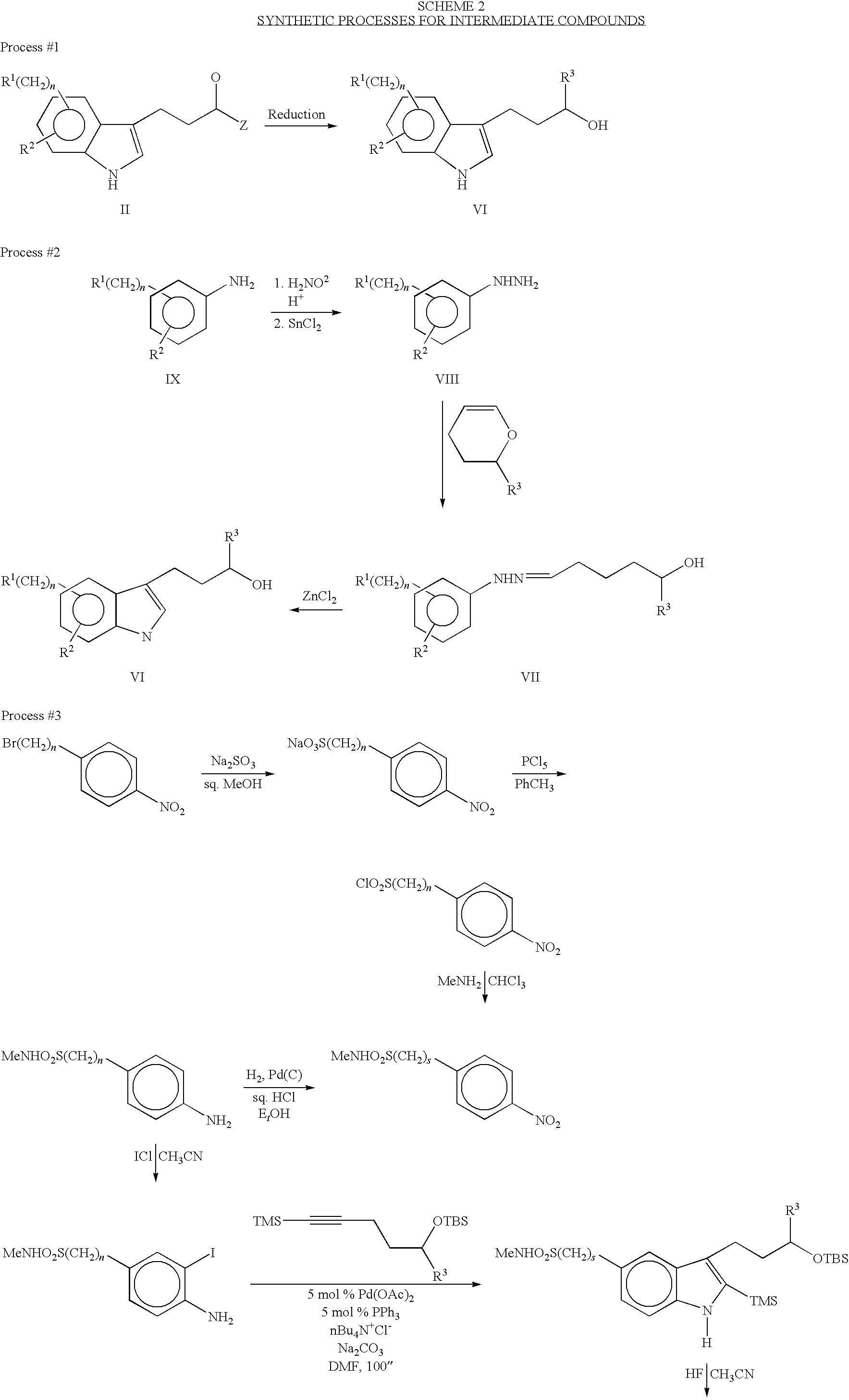

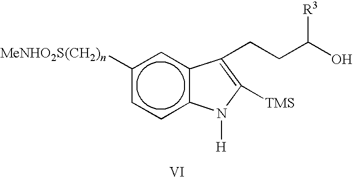

Scheme 2 sets out several processes for synthesis of Formula VI intermediates. Again the skilled synthetic chemist would know how to adapt these processes for preparation of specific Formula VI compounds. Process #1 symbolizes reduction processes in general for conversion of the compound II carbonyl moiety to the alcohol. Selection of reaction parameters would of course be dictated by the reactivity of R¹. The symbol Z is either lower alkyl, lower alkoxy or hydroxy. When Z is lower alkyl, R³ in intermediate product VI will be lower alkyl.

A modification of process 1 can be done in conjunction with Scheme 1 by combining an appropriate compound of Formula II with a pyrimidinylpiperazine intermediate of Formula III followed by sodium cyanoborohydride treatment to produce the desired Formula I product. This is a desirable option for synthesizing products wherein R³ is lower alkyl and R¹ is a moiety that is chemically inert to sodium cyanoborohydride.

Process #2 comprises synthetic elaboration of the indolylalkyl alcohol via the hydrazone intermediates VII and VIII followed by ring-closure to the indole system.

Process #3 sets out a particularly useful method for preparation of Formula VI intermediates wherein R¹ is —SO₂ NR⁷R⁸. Specific examples comprising process #3 are given in detail in the experimental section, infra.

Additional formula I products can also be obtained by chemical conversions of the R¹-indolylsubstituent. Examples comprise conversion of the cyano group to the aminocarbonyl substituent by the action of strong hydroxide reagents such as KOH; conversion of amino groups to amides, lactams and sulfonamides as well as numerous conversions of an R¹ hydroxy substituent to other functional groups as shown in Scheme 4.

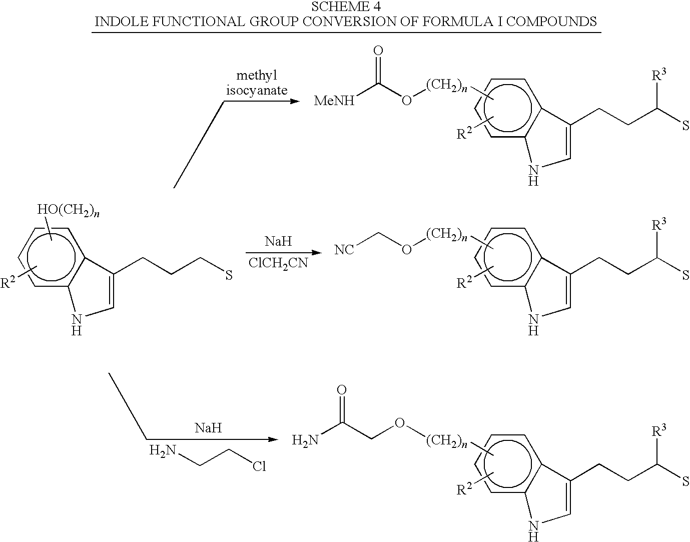

Reagents, solvents, and reaction conditions for the above described steps of these processes would be familiar to one skilled in organic synthesis as the processes comprise standard organic reactions, the details of which are readily available in the chemical literature. These processes may be adapted to variation in order to produce other compounds embraced by this invention but not specifically disclosed. Variations of the methods to produce the same compounds in somewhat different fashion will also be evident to one skilled in the art.

To provide greater descriptive detail, representative synthetic examples are provided hereinbelow in the “Description of Specific Embodiments” section.

Serotonin has been linked to the pathophysiology of anxiety, depression, and sexual dysfunction as noted above. Moreover, more specific serotoninergic agents, which would treat the anxiety, depression, or sexual dysfunction without all of the other actions, are potentially useful. Among the most promising are compounds with selectivity for the 5-HT₁D sub-type of serotonin receptors. In this regard the compounds of the instant invention demonstrate potent affinity and agonist activity at the 5-HT₁D site. Formula I compounds of interest have potencies wherein IC₅0 values of these compounds are less than 100 nmolar. Preferred compounds have IC₅0 values below 10 nmolar.

Determination of 5-HT₁D binding properties was accomplished employing methodology such as that described by Heuring and Peroutka, J. Neurosci., 7(3), 1987, 894-903; with only minor modifications. In vitro IC₅0 (nM) test values were determined for the compounds of this invention employing tritiated serotonin.

Another aspect of the instant invention provides a method for treating a anxiety, depression, or sexual dysfunction sufferer which comprises systemic administration to the sufferer of a therapeutically effective amount of a compound of Formula I or a pharmaceutically acceptable salt and/or solvate thereof, or a metabolite thereof. Compounds of Formula XXI encompass additional compounds relating to Formula I compounds in that R³ can be hydrogen regardless of the nature of R¹. Compounds of XXI are defined as

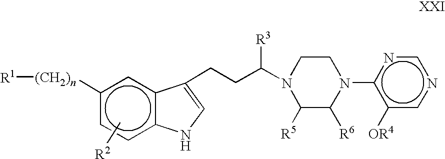

wherein R¹ is a substituent selected from the group consisting of hydrogen, halogen, lower alkyl, lower alkoxy, R⁷-substituted phenyl-lower alkoxy, amino, cyano, hydroxy, nitro, —OCH₂CN, —OCH₂CONR⁷R⁸, —SO₂ NR⁷R⁸, —O₂CR⁹, —SO₂R⁹, —O₂CNR⁷R⁸, —COR⁸, —CO₂R⁹, —CONR⁷R⁸, NR⁷CO₂R⁹, —NR⁷COR⁸, —NR⁷SO₂R⁹ and

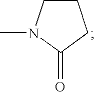

R² is selected from hydrogen, halogen, lower alkyl, lower alkoxy and —CO₂R⁹;
R³, R⁵, R⁶ and R⁷ are independently selected from hydrogen and lower alkyl;
R⁴ is lower alkyl;
R⁸ is selected from hydrogen, lower alkyl, R⁷-phenyl-lower alkyl and trifluoromethyl;
R⁹ is selected from lower alkyl and R⁷-phenyl-lower alkyl; and
n is zero or the integers 1 or 2.

The administration and dosage regimen of compounds of Formula XXI is considered to be done in the same manner as for the reference compound sumatriptan, cf: Oxford, GB 2,162,522A. Although the dosage and dosage regimen must in each case be carefully adjusted, utilizing sound professional judgment and considering the age, weight and condition of the recipient, the route of administration and the nature and gravity of the illness, generally the daily dose will be from about 0.05 to about 10 mg/kg, preferably 0.1 to 2 mg/kg, when administered parenterally and from about 1 to about 50 mg/kg, preferably about 5 to 20 mg/kg, when administered orally. In some instances, a sufficient therapeutic effect can be obtained at lower doses while in others, larger doses will be required. Systemic administration refers to oral, intra-nasal, rectal and parenteral (i.e. intramuscular, intravenous and subcutaneous). Intracisternal, intravaginal, intraperitoneal, sublingual, topical, and bucal administration are also within the scope of the present invention. Generally, it will be found that when a compound of the present invention is administered orally, a larger quantity of the active agent is required to produce the same effect as a smaller quantity given intra-nasally or parenterally. In accordance with good clinical practice, it is preferred to administer the instant compounds at a concentration level that will produce effective results without causing any harmful or untoward side effects.

The compounds of the present invention may be administered for treatment of anxiety, depression, or sexual dysfunction either as individual therapeutic agents or as mixtures with other therapeutic agents. Therapeutically, they are generally given as pharmaceutical compositions comprised of an effective amount of a compound of Formula I or a pharmaceutically acceptable salt thereof and a pharmaceutically acceptable carrier. Pharmaceutical compositions which provide from about 1 to 500 mg of the active ingredient per unit dose are preferred and are conventionally prepared as tablets, lozenges, capsules, powders, aqueous or oily suspensions, syrups, elixirs, and aqueous solutions. The compositions may be given in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist. Moreover, the patient in need of treatment may concurrently suffer from one or more disorders, including anxiety, depression, obesity, drug abuse/addiction, alcohol abuse, sleep disorders, TIC disorder, and behavioral/cognitive symptoms of Alzheimer's disease.

The nature of the pharmaceutical composition employed will, of course, depend on the desired route of administration. For example, oral compositions may be in the form of tablets or capsules and may contain conventional excipients such as binding agents (e.g. starch) and wetting agents (e.g. sodium lauryl sulfate). Solutions or suspensions of a Formula I compound with conventional pharmaceutical vehicles are employed for intra-nasal and parenteral compositions such as an aqueous solution for intravenous injection or an oily suspension for intramuscular injection.

### DETAILED DESCRIPTION OF THE PREFERRED EMBODIMENTS

The compounds which constitute this invention, their methods of preparation and their biologic actions will appear more fully from consideration of the following examples, which are given for the purpose of illustration only and are not be construed as limiting the invention in sphere or scope. In the following examples, used to illustrate the foregoing synthetic processes, temperatures are expressed in degrees Celsius and melting points are uncorrected. The nuclear magnetic resonances (NMR) spectral characteristics refer to chemical shifts (δ) expressed as parts per million (ppm) versus tetramethylsilane (TMS) as reference standard. The relative area reported for the various shifts in the ¹H NMR spectral data corresponds to the number of hydrogen atoms of a particular functional type in the molecule. The nature of the shifts as to multiplicity is reported as broad singlet (bs), singlet (s), multiplet (m), triplet (t) or doublet (d). Abbreviations employed are DMSO-d₆ (deuterodimethylsulfoxide), CDCl₃ (deuterochloroform) and are otherwise conventional. The infrared (IR) spectral descriptions include only absorption wave numbers (cm⁻¹) having functional group identification value. The IR determinations were employed using potassium bromide (KBr) as diluent. The elemental analyses are reported as percent by weight.

The following examples describe in detail the preparation of compounds of Formula I, as well as synthetic intermediates in each process. It will be apparent to those skilled in the art that modifications, both of materials and methods, will allow preparation of other compounds disclosed herein. From the foregoing description and the following examples it is believed that one skilled in the art is able to use the invention to the fullest extent.

### A. Preparation of Intermediate Compounds

Some representative procedures for preparation of synthetic intermediate compounds utilized in the three processes of Scheme 2 are given hereinbelow. Most starting materials and certain intermediates (e.g. Formula II and V compounds), are either commercially available or procedures for their synthesis are readily available in the chemical literature allowing their full utilization by one skilled in the art of organic synthetic chemistry.

### Compounds of Formula VI

### Compounds of Type:

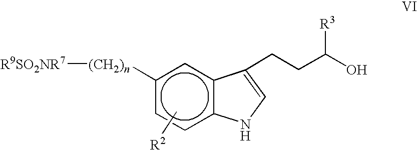

### Example 1

### 3-(5-Ethanesulfonylamino-1H-indol-3-yl)propanol

### 5-Ethanesulfonylamino-1H-indole

To a solution of 5-amino-1H-indole (10.0 g, 76 mmol) and triethylamine (15.8 mL, 114 mmol) in 100 mL CH₂Cl₂ at 0 degree C. was added dropwise a solution of ethanesulfonyl chloride (7.9 mL, 83 mmol) in 25 mL of CH₂Cl₂. The solution was allowed to slowly warm to 23 degree C. over 20 h. The reaction mixture was concentrated in vacuo and the residue dissolved in 400 mL of ethyl acetate. The organic layer was washed with 100 mL of water, 50 mL of 0.1M HCl, 50 mL of saturated NaHCO₃ solution, and 50 mL of saturated NaCl solution. The organic layer was dried over anhydrous K₂CO₃, filtered, and concentrated in vacuo to obtain 5-ethanesulfonylamino-1H-indole (16.9 g, >99%) which was used without further purification.

### 5-Methyl(ethanesulfonyl)amino-1H-indole

To a solution of 5-ethane-sulfonamino-1H-indole (8.96 g, 40 mmol) of in 200 mL of anhydrous THF at 0 degree C. was added dropwise a 2.5M solution of n-BuLi in hexane (17.6 mL, 44 mmol). After stirring for 30 minutes at 0 degree C., methyl iodide (6.25 g, 44 mmol) was added dropwise. The reaction mixture was allowed to warm to 23 degree C. and stir for 66 h. The mixture was poured into ethyl acetate, washed with five 100 mL portions of 1N NaOH, and, finally, with a saturated NaCl solution. The organic layer was dried over anhydrous K₂CO₃, filtered, and concentrated in vacuo to obtain 5-methyl(ethanesulfonyl)amino-1H-indole (9.52 g, >99%).

### 3-(5-Ethanesulfonylamino-1H-indol-3-yl)propanoic acid

A solution of 5-ethanesulfonylamino-1H-indole (8.0 g, 36 mmol), acrylic acid (5.15 g, 71 mmol), and acetic anhydride (7.3 g, 71 mmol) in 35 mL of acetic acid was heated at 90° C. for 20 h. The volatile materials were removed under high vacuum at 90° C. The remaining residue was dissolved in 1N NaOH and subsequently acidified to pH 1 with concentrated HCl. The aqueous solution was then extracted with five portions of ethyl acetate. The organic extracts were combined, dried over anhydrous MgSO₄, filtered, and concentrated in vacuo to yield 3-(5-ethanesulfonylamino-1H-indol-3-yl)propanoic acid (10.6 g of crude material). NMR and mass spectral analysis indicated the presence of the desired product. No attempt was made to purify this material; it was immediately subjected to reduction as described below.

### 3-(5-Ethanesulfonylamino-1H-indol-3-yl)propanol

To a solution of crude 3-(5-ethanesulfonylamino-1H-indol-3-yl)propanoic (10.6 g, 36 mmol) in 50 mL of THF at 0° C. was added a solution of 1.0M borane in THF (161 mL, 161 mmol). The reaction mixture was allowed to warm to 23° C. and stand for two h. The reaction was cooled to 0° C. and 100 mL of 5N KOH was added slowly. After standing for 16 h, the organic layer was separated and the aqueous phase extracted with four portions of THF. The aqueous phase was neutralized with concentrated HCl and extracted with ethyl acetate. The combined organic layers were dried over anhydrous K₂CO₃, filtered, and concentrated in vacuo. Silica gel chromatography (95:5:0.5 CH₂Cl₂:MeOH:NH₄₀H) of the residue afforded 3-(5-ethanesulfonylamino-1H-indol-3-yl)propanol (1.76 g, 18%)

### Compounds of Type

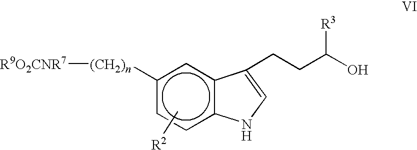

### Example 2

### 3-[5-(Phenylmethoxycarbonyl)amine]-1H-indol-3-yl]propanol

### Phenylmethyl (1H-indol-5-yl)carbamate

To a solution of 5-aminoindole (10.0 g, 76 mmol) and triethylamine (7.67 g, 76 mmol) in acetonitrile (400 mL) at 0° C. was added dropwise a solution of carboxybenzyloxychloride (12.93 g, 75.8 mmol) in acetonitrile (80 mL). After the addition was complete the reaction was allowed to warm to 23° C. and stand for 60 h. The solvent was removed in vacuo and the residue treated with water containing Na₂CO₃ (76 mmol). The mixture was extracted with four portions of CH₂Cl₂. The combined organic extracts were washed with a saturated NaCl solution, dried with K₂CO₃, filtered, and concentrated in vacuo. Silica gel chromatography (4:1 to 1:1 hexane:ethyl acetate gradient) of the concentrate afforded phenylmethyl-(1H-indol-5-yl)carbamate (5.11 g, 25%).

### Phenylmethyl [3-[(dimethylamino)methyl]-1H-indol-5-yl)carbamate

To a solution of phenylmethyl (1H-indol-5-yl)carbamate (3.76 g, 14.1 mmol) in ethanol (6 mL) was added dimethylamine (1.75 mL, 40% aq solution) and formaldehyde (1.25 mL, 40% aq solution). The reaction was heated at reflux for 16 h. The solvent was removed in vacuo and the residue treated with 10% Na₂CO₃ solution and extracted with four portions of ethyl acetate. The combined organic extracts were washed with saturated NaCl solution, dried with Na₂CO₃, filtered, and concentrated in vacuo. Silica gel chromatography (93:7:0.7 to 90:10:1 CH₂Cl₂:MeOH:NH₄ OH gradient) of the concentrate afforded phenylmethyl [3-[(dimethylamino)methyl]-1H-indol-5-yl]carbamate (2.30 g, 51%).

### N,N,N-Trimethyl-1-[5-[(phenylmethoxycarbonyl)amine]-1H-indol-3-yl]methanaminium iodide

To a solution of phenylmethyl[3-[(dimethylamino)-methyl]-1H-indol-5-yl]carbamate (2.30 g, 7.1 mmol) in THF (75 mL) at 0° C. was added dropwise methyl iodide (1.52 g, 10.7 mmol). The reaction was allowed to warm to 23° C. and stand for 2 h. The precipitate was filtered and dried in vacuo to afford N,N,N-trimethyl-1-[5-[(phenylmethoxycarbonyl)amine]-1H-indol-3-yl]methanaminium iodide (2.94 g, 89%).

### Methyl 2-methoxycarbonyl-3-[5-[(phenylmethoxycarbonyl)amine]-1H-indol-3-yl]propionate

To a solution of sodium dimethyl malonate (1.12 g, 7.3 mmol) in MeOH (15 mL) was added a MeOH solution (50 mL) containing N,N,N-trimethyl-1-[5-](phenylmethoxycarbonyl)amine]-1H-indol-3-yl]methanaminium iodide. The resulting solution was heated at reflux for 24 h. The solvent was removed in vacuo. The concentrate was treated with saturated NaHCO₃ and extracted with three portions of ethyl acetate. The combined organic extracts were washed with a saturated NaCl solution, dried with K₂CO₃, filtered, and concentrated in vacuo. Silica gel chromatography (60:40 hexane:ethyl acetate) of the concentrate afforded methyl 2-methoxycarbonyl-3-[5-[(phenylmethoxycarbonyl)amine]-1H-indol-3-yl]propionate (1.17 g, 59%) whose structure was confirmed by NMR and MS analysis.

### Methyl 3-[5-[(phenylmethoxycarbonyl)amine]-1H-indol-3-yl]propionate

To a solution of methyl 2-methoxycarbonyl-3-[5-[(phenylmethoxycarbonyl)amine]-1H-indol-3-yl]-propionate (1.17 g, 2.8 mmol) in pyridine (15 mL) was added sodium iodide (0.83 g, 5.5 mmol) and the resulting solution heated at reflux for 24 h. The solvent was removed in vacuo. The concentrate was treated with saturated NaHCO₃ and extracted with four portions of ethyl acetate. The combined organic extracts were washed with a saturated NaCl solution, dried with K₂CO₃, filtered, and concentrated in vacuo. Silica gel chromatography (70:30 hexane:ethyl acetate) of the concentrate afforded methyl 3-[5-[(phenylmethoxycarbonyl)amine]-1H-indol-3-yl]propionate (0.47 g, 48%) whose structure was confirmed by NMR and MS analysis.

### 3-[5-[(Phenylmethoxycarbonyl)amine]-1H-indol-3-yl]propanol

To a suspension of LiAlH₄ (0.08 g, 2 mmol) in THF at −20° C. was added dropwise a THF solution (3 mL) of methyl 3-[5-[(phenyl-methoxycarbonyl]amine]-1H-indol-3-yl]propionate (0.47 g, 1.3 mmol). After the addition was complete, the reaction was allowed to stand at −20° C. for 5 h. The excess reducing agent was destroyed by the dropwise addition of water (0.1 mL), followed by 15% NaOH (0.1 mL), and, finally, 0.3 mL of water. The mixture was allowed to warm to 23° C. and stand for 16 h. The reaction was filtered through a pad of celite and the filter cake washed with Et₂O. The combined organic phases were dried with K₂CO₃, filtered, and concentrated in vacuo. Silica gel chromatography (1:1 hexane:ethyl acetate) of the concentrate afforded 3-[5-[(phenylmethoxycarbonyl)amine]-1H-indol-3-yl]propanol (0.31 g, 72%) whose structure was confirmed by NMR and MS analysis.

### Compounds of Type:

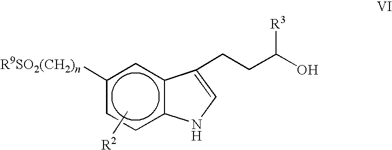

### Example 3

### 3-[5[-(Methylsulfonyl)methyl]-1H-indol-3-yl]propanol 1-[2-(Methylsulfonyl)methyl]-4-nitrobenzene

A solution of 4-nitrobenzyl bromide (2.16 g, 10 mmol) and sodium methanesulfinate (1.12 g, 11 mmol) in DMF (25 mL) was heated at reflux for 0.5 h. The solvent was removed in vacuo and the residue extracted with CH₂Cl₂ and water. The combined organic phases were washed with a saturated NaCl solution, dried over MgSO₄, filtered, and concentrated in vacuo to yield 1-[2-(methylsulfonyl)methyl]-4-nitrobenzene (1.54 g, 71.6%) which was used without further purification.

### 1-8 2-(Methylsulfonyl)ethyl]-4-nitrobenzene

This compound was prepared in a similar manner from 1-(2-bromoethyl)-4-nitrobenzene and sodium methanesulfinate.

### 4-Amino-1-[2-(methylsulfonyl)methyl]benzene

A suspension of 1-[2-(methylsulfonyl)methyl]-4-nitrobenzene (27 g, 126 mmol) and concentrated HCl (5 mL) in 300 mL of 66% ethanol (aq) was hydrogenated (50 psi) over 10% Pd/C catalyst (4 g) at 23° C. for 16 h. The catalyst was removed by filtration and the filtrate concentrated in vacuo to remove the ethanol. The remaining aqueous phase was made basic to litmus by addition of 50% NaOH whereupon the product, 4-amino-1-[2-(methylsulfonyl)methyl]benzene, precipitated (21.54 g, 93%).

### 1-[2-(5-Hydroxypentylidene)hydrazinyl]-4-[(methylsulfonyl)methyl]benzene

To a concentrated HCl solution (40 mL) of 4-amino-1-[(methylsulfonyl)methyl]benzene (8.0 g, 43 mmol) was added water (40 mL). The reaction was cooled to 0° C. and a solution containing NaNO₃ (3.58 g, 51 mmol) in water (10 mL) was added dropwise. The reaction was allowed to stir at 0° C. for one h and then poured into a solution of SnCl₂ (48.78 g, 216 mmol) in 6N HCl (160 mL) at −40° C. The reaction was allowed to warm to 23° C. and the pH then adjusted to 2 with 50% NaOH. Ethanol (300 mL) and dihydropyran (4.73 g, 56 mmol) were added and the reaction was stirred for 16 h. The mixture was made basic with 50% NaOH (pH=10) and then filtered over celite. The resulting solution was concentrated in vacuo and the remaining aqueous phase extracted with ethyl acetate. The combined organic layers were washed with water, dried with MgSO₄, filtered, and concentrated in vacuo. Silica gel chromatography (CH₂Cl₂:MeOH:NH₄ OH; 95:5:0.5) of the concentrate afforded the desired hydrazone (4.34 g, 38%).

### 3-[5-[(Methylsulfonyl)methyl]-1H-indol-3-yl]propanol

A 1,2-dimethoxyethane solution containing 1-[2-(5-hydroxy-pentylidene)hydrazinyl]-4-[(methylsulfonyl)methyl]benzene (4.74 g, 17 mmol) and ZnCl₂ (11.4 g, 84 mmol) was heated at reflux for 24 h. The reaction was concentrated in vacuo and the residue dissolved in water and extracted with ethyl acetate. The combined organic layers were washed with water, dried with MgSO₄, filtered, and concentrated in vacuo. Silica gel chromatography (CH₂Cl₂ MeOH:NH₄ OH; 95:5:0.5) of the concentrate afforded 3-[5-[(methylsulfonyl)methyl]-1H-indol-3-yl]propanol (1.23 g, 28%).

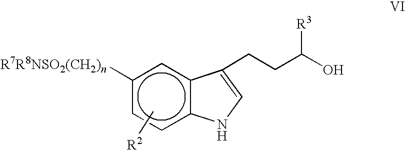

### Example 4

### 3-(3-Hydroxypropyl)-N-methyl-1H-indole-5-methane sulfonamide

### 4-[2-(5-Hydroxypentylidene)hydrazinyl]-N-methylbenzene methanesulfonamide

To a suspension of 14.0 g (69.9 mmol, 1.0 equiv.) of 4-((N-methyl methanesulfonamido)methyl)aniline in 140 mL of concentrated aqueous hydrochloric acid and 70 mL of water at 0° C. was added 4.82 g of sodium nitrite in 70 mL of water. The mixture was stirred for fifteen minutes at 0° C. Meanwhile a solution of 78.9 g of stannous chloride in 140 mL of concentrated aqueous hydrochloric acid was prepared and cooled to −45° C. The solution of the diazonium salt was filtered into the stannous chloride solution. The reaction was allowed to warm to 0° C. over the course of one hour and then carefully brought to pH 3.5 by the addition of solid potassium hydroxide. The reaction was diluted with 420 mL of ethanol and 7.65 mL (83.9 mmol, 1.2 equiv.) of 3,4-dihydro-2H-pyran was added. The reaction mixture was allowed to stir at room temperature for sixteen hours. The pH was further raised to 10 by the addition of solid potassium hydroxide. The mixture was filtered and ethanol was removed in vacuo. The remaining aqueous layer was extracted three times with 500 mL portions of ethyl acetate. The combined organic layers were dried over anhydrous sodium sulfate, filtered and concentrated to afford an oil. The oil was chromatographed on silica gel using 5% methanol in methylene chloride containing 0.5% concentrated aqueous ammonium hydroxide to obtain 7.85 g (38%) of 4-[2-(5-hydroxypentylidene) hydrazinyl]-N-methylbenzenemethane sulfonamide.

### 3-(3-Hydroxypropyl)-N-methyl-1H-indole-5-methanesulfonamide

To a solution of 23.6 g (173 mmol, 5.0 equiv.) of zinc chloride in 1000 mL of anhydrous 1,2-dimethoxyethane under a nitrogen atmosphere was added a solution of 10.37 g (34.65 mmol, 1.0 equiv.) of 4-[2-(5-hydroxypentylidene)hydrazinyl]-N-methylbenzenemethanesulfonamide in 200 mL of anhydrous 1,2-dimethoxyethane. The solution was heated to reflux for thirty hours. The reaction was cooled to room temperature and the volume concentrated to 500 mL in vacuo. The residue was diluted with 1000 mL of ethyl acetate and washed with 200 mL portions of water and saturated aqueous sodium chloride. The organic layer was dried over magnesium sulfate, filtered and concentrated in vacuo to yield an oil. The oil was purified by chromatography on silica gel using 5% methanol in methylene chloride containing 0.5% concentrated aqueous ammonium hydroxide to obtain 5.16 g (53%) of 3-(3-hydroxypropyl)-N-methyl-1H-indole-5-methanesulfonamide.

### Example 5

### Alternate Synthesis

### Via Scheme 2 Process #3

### 4-Amino-3-iodo-N-methylbenzenemethanesulfonamide

To a suspension of 1.06 g (5.31 mmol, 1.0 equiv.) of 4-amino-N-methylbenzenemethanesulfonamide in 20 mL of acetonitrile was added 0.862 g (5.31 mmol, 1.0 equiv.) of iodine monochloride. The reaction was stirred for 15 minutes at room temperature. The mixture was partitioned between 25 mL of ethyl acetate and 15 mL of 20% aqueous sodium thiosulfate. The organic layer was dried over anhydrous sodium sulfate, filtered and concentrated in vacuo to yield an oil. The oil was filtered through a plug of silica gel using 40% ethyl acetate in hexane to yield 1.13 g (65%) of 4-amino-3-iodo-N-methylbenzenemethanesulfonamide

### 1-t-Butyldimethylsilyloxy-4-pentyne

To a suspension of 18.0 g (749 mmol, 1.05 equiv.) of sodium hydride in 500 mL of THF at 0° C. was added a solution of 60.0 g (713 mmol, 1.00 equiv.) of 4-pentyne-1-ol in 150 mL of THF. When the addition was complete the reaction was allowed to warm to room temperature over one hour. To this mixture was added 113 g (749 mmol, 1.05 equiv.) of t-butyldimethylsilyl chloride in 150 mL of THF. The reaction was allowed to stir for 16 hours at room temperature then diluted with 1200 mL of hexane. The organic layer was washed with 500 mL of saturated aqueous sodium bicarbonate, dried over anhydrous sodium sulfate and concentrated in vacuo. The resulting liquid was distilled at 39°-42° C. at 0.05 mm Hg to yield 135 g (96%) of 1-t-butyldimethylsilyloxy-4-pentyne

### 1-t-Butyldimethylsilyloxy-5-trimethylsilyl-4-pentyne

To a solution of 135 g (682 mmol, 1.0 equiv.) of 1-t-butyldimethylsilyloxy-4-pentyne in 700 mL of THF at −78° C. was added 311 mL (716 mmol, 1.05 equiv.) of a 2.3M solution of n-butyllithium in hexane. The −78° C. bath was removed and the reaction temperature was monitored as it rose to 0° C. The reaction was recooled to −78° C. and 90.8 mL (716 mmol, 1.05 equiv.) of trimethylsilyl chloride was added dropwise. The reaction was then allowed to warm slowly in the cold bath for 16 hours. The reaction was diluted with 2000 mL of hexane, washed with 500 mL of saturated aqueous sodium bicarbonate, dried over anhydrous sodium sulfate and concentrated in vacuo. The resulting liquid was distilled at 71°-74° C. at 0.05 mm Hg to yield 168 g (91%) of 1-t-butyldimethylsilyloxy-5-trimethylsilyl-4-pentyne.

### 3-(3-t-Butyldimethylsilyloxypropyl)-2-trimethylsilyl-N-methyl-1H-indole-5-methanesulfonamide

To a solution of 0.326 g (1.0 mmol, 1.0 equiv.) of 4-amino-3-iodo-N-methylbenzenemethanesulfonamide in 20 mL of dimethylformamide was added 0.541 g (2.0 mmol, 2.0 equiv.) of 1-t-butyldimethylsilyloxy-5-trimethylsilyl-4-pentyne, 0.278 g (1.0 mmol, 1.0 equiv.) of tetrabutylammonium chloride, 0.530 g (5.0 mmol, 5.0 equiv.) of sodium carbonate, 0.0131 g (0.05 mmol, 0.05 equiv.) of triphenylphosphine, and 0.0112 g (0.05 g, 0.05 equiv.) of palladium (II) acetate. The mixture was heated in a 100° C. oil bath for 16 hours. The mixture was cooled to room temperature and the dimethylformamide removed in vacuo. The residue was diluted with 75 mL of ethyl acetate and washed with 25 mL of saturated aqueous sodium bicarbonate. The organic layer was dried over anhydrous sodium sulfate, filtered and concentrated in vacuo to yield an oil. The oil was filtered through a plug of silica gel with 25% ethyl acetate in hexane to yield 0.411 g (88%) of 3-(3-t-butyldimethylsilyloxypropyl)-2-trimethylsilyl-N-methyl-1H-indole-5-methanesulfonamide.

### 3-(3-Hydroxypropyl)-2-trimethylsilyl-N-methyl-1H-indole-5-methanesulfonamide

To a solution of 0.0738 g (0.157 mmol, 1.0 equiv.) of 3-(3-t-butyldimethysilyloxypropyl)-2-trimethylsilyl-N-methyl-1H-indole-5-methanesulfonamide in 2 mL of pyridine at 0° C. was added 1 mL of 48% aqueous hydrofluoric acid. The solution was stirred for 20 minutes at 0° C. The solution was diluted with 25 mL of ethyl acetate and washed with 10 mL of 10% aqueous sodium carbonate. The organic layer was dried over anhydrous sodium sulfate, filtered and concentrated in vacuo to yield 0.0551 g (99%) of 3-(3-hydroxypropyl)-2-trimethylsilyl-N-methyl-1H-indole-5-methanesulfonamide, homogeneous by ¹H NMR and TLC.

### 3-(3=Hydroxypropyl)-N-methyl-1H-indole-5-methanesulfonamide

To a solution of 0.0551 g (0.155 mmol, 1.0 equiv.) of 3-(3-hydroxypropyl)-2-trimethylsilyl-N-methyl-1H-indole-5-methanesulfonamide in 2 mL of methylene chloride at 0° C. was added 0.01 mL (0.155 mmol, 1.0 equiv.) of trifluoroacetic acid. After 15 minutes the reaction was allowed to warm to room temperature. After a total of thirty minutes the reaction was poured into 15 mL of ethyl acetate and washed with 5 mL of saturated aqueous sodium bicarbonate. The organic layer was dried over anhydrous sodium sulfate, filtered and concentrated in vacuo to yield 0.0413 g (95%) of 3-(3-hydroxypropyl)-N-methyl-1H-indole-5-methanesulfonamide, homogeneous by ¹H NMR and TLC.

### Example 6

### 3-(3-Hydroxypropyl)-N—N-dimethyl-1H-indole-5-methanesulfonamide

### Sodium 4-nitrobenzyl sulfonate

A suspension of 100 g (463 mmol, 1.0 equiv.) of 4-nitrobenzyl bromide and 64.2 g (509 mmol, 1.1 equiv.) of sodium sulfite in 500 mL of methanol and 500 mL of water was heated to reflux. The progress of the reaction was monitored by TLC. When the bromide was consumed, the reaction mixture was allowed to cool to room temperature. The product precipitated and was collected by filtration. It was thoroughly dried for sixteen hours at 65° C. at 0.05 mm Hg to give 100 g (90%) of sodium 4-nitrobenzyl sulfonate.

### 4-Nitrobenzyl sulfonyl chloride

To a suspension of 28.3 g (118 mmol, 1.0 equiv.) of finely ground sodium 4-nitrobenzyl sulfonate in 250 mL of toluene was added 24.6 g (118 mmol, 1.0 equiv) of finely ground phosphorous pentachloride as the solid in portions. After addition the mixture was heated to reflux for one hour. The mixture was cooled, and volatile material was removed in vacuo. The residue was dissolved in 500 mL chloroform and 250 mL of water. The organic layer was separated, dried over anhydrous sodium sulfate, filtered and concentrated to give 27.8 g (100%) of 4-nitrobenzyl sulfonyl chloride, pure by ¹H NMR.

### 4-Nitro-N,N-dimethylbenzenmethanesulfonamide

Anhydrous dimethylamine was bubbled through 250 mL of chloroform at 0° C. for thirty minutes. A solution of 27.8 g (118 mmol, 1.0 equiv.) of 4-nitrobenzyl sulfonyl chloride was added dropwise at 0° C. An exothermic reaction ensued and the mixture was allowed to warm to room temperature. The mixture was concentrated in vacuo to yield 32.2 g of 4-nitro-N,N-dimethylbenzenemethanesulfonamide contaminated with dimethylamine hydrochloride. This mixture was taken on without purification.

### 4-Amino-N,N-dimethylbenzenemethanesulfonamide

To a solution of 22.3 g of crude 4-nitro-N,N-dimethyl-benzenemethanesulfonamide (contaminated with dimethylamine hydrochloride as described above) in 45 mL of 2N hydrochloric acid, 100 mL of ethanol and 200 mL of water was added 5.0 g of 10% palladium on carbon. The mixture was hydrogenated in a Parr apparatus at 60 to 50 psi for three hours. The mixture was filtered and the ethanol removed in vacuo. The resulting aqueous solution was treated with 15% aqueous potassium hydroxide. Precipitate began forming at pH four. Addition of potassium hydroxide was continued until the pH had reached nine. The product was isolated by filtration and dried for sixteen hours at 65° C. at 0.05 mm Hg to give 21.0 g (83%, two steps) of 4-amino-N,N-dimethylbenzenemethanesulfonamide.

### 4-Hydrazinyl-N,N-dimethylbenzenemethanesulfonamide hydrochloride

To a suspension of 4.30 g (20.1 mmol, 1.0 equiv.) of 4-amino-N,N-dimethylbenzenemethanesulfonamide in 40 mL of concentrated aqueous hydrochloric acid and 20 mL of water cooled to 0° C. was added a solution of 1.38 g (20.1 mmol, 1.0 equiv.) of sodium nitrite in 20 mL of water dropwise. The reaction mixture became homogeneous during the addition. The reaction was stirred for fifteen minutes at 0° C. after the addition was complete. Meanwhile a solution of 22.6 g (100 mmol, 5.0 equiv.) of stannous chloride dihydrate in 40 mL of concentrated aqueous hydrochloric acid was prepared and cooled to −40° C. The solution of the diazonium salt was filtered into the stannous chloride solution. The reaction was allowed to warm to room temperature over the course of one hour. The reaction was then recooled to 0° C. and the precipitated product was collected by filtration. The solid was dried at 65° C. at 0.05 mm Hg for 45 minutes to yield 5.02 g (94%) of 4-hydrazinyl-N,N-dimethylbenzenemethanesulfonamide hydrochloride.

### 4-[2-(5-Hydroxypentylidene)hydrazinyl]-N,N-dimethylbenzenemethanesulfonamide

To a solution of 5.02 g (18.9 mmol, 1.0 equiv.) of 4-hydrazinyl-N,N-dimethyl-benzenemethanesulfonamide in 20 mL of water and 20 mL of ethanol was added sufficient sodium acetate to raise the pH to four. To the resulting slurry was added 1.90 mL (20.8 mmol, 1.1 equiv.) of 3,4-dihydro-2H-pyran. The reaction was stirred for sixteen hours then diluted with 200 mL of ethyl acetate. The organic layer was washed with 10% aqueous potassium carbonate, dried over anhydrous sodium sulfate and concentrated in vacuo to yield an oil. Chromatography on silica gel using 5% methanol in methylene chloride containing 0.5% concentrated aqueous ammonium hydroxide yielded 1.67 g (28%) of pure 4-[2-(5-hydroxypentylidene)hydrazinyl]N,N-dimethylbenzenemethanesulfonamide as an oil.

### 3-(3-Hydroxypropyl)-N,N-dimethyl-1H-indole-5-methanesulfonamide

To a solution of 3.63 g (26.6 mmol, 5.0 equiv.) of zinc chloride in 150 mL of anhydrous 1,2-dimethoxyethane under a nitrogen atmosphere was added a solution of 1.67 g (5.33 mmol, 1.0 equiv.) of 4-[2-(5-hydroxypentylidene)hydrazinyl]-N,N-dimethylbenzenemethanesulfonamide in 50 mL of anhydrous 1,2-dimethoxyethane. The solution was heated to reflux for sixteen hours. The reaction was cooled to room temperature and the volume concentrated to 25 mL in vacuo. The residue was diluted with 100 mL of ethyl acetate and washed with 20 mL portions of water and saturated aqueous sodium chloride. The organic layer was dried over anhydrous sodium sulfate, filtered and concentrated in vacuo to yield an oil. The oil was purified by chromatography on silica gel using 5% methanol in methylene chloride containing 0.5% concentrated aqueous ammonium hydroxide to obtain 0.409 g (26%) of 3-(3-hydroxypropyl)-N,N-dimethyl-1H-indole-5-methanesulfonamide.

### Compounds of Type:

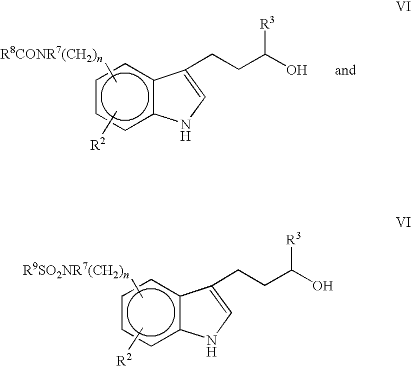

### Example 7

### N-[[3-(3-Hydroxypropyl)-1H-indol-5-yl]methyl]methanesulfonamide

### N-[(4-Nitrophenyl)methyl]methanesulfonamide

To a solution of 25.0 g (133 mmol, 1.0 equiv.) of 4-nitro benzylamine hydrochloride in 500 mL of methylene chloride at 0° C. was added 46.4 mL (331 mmol, 2.5 equiv.) of triethylamine followed by 11.3 mL (146 mmol, 1.1 equiv.) of methanesulfonyl chloride dropwise. The reaction was stirred for thirty minutes at 0° C. then diluted with 500 mL of chloroform. The organic layer was washed with 150 mL portions of 1N HCl and saturated sodium chloride. The organic layer was dried over magnesium sulfate, filtered and concentrated to give 25.8 g (85%) of N-[(4-nitrophenyl)methyl]methanesulfonamide.

### N-[(4-Aminophenyl)methyl]methanesulfonamide

To a suspension of 25.9 g (113 mmol, 1.0 equiv.) or N-[(4-nirropnenyl)mernyl]methanesulfonamide in 113 mL of 1N HCl (113 mmol, 1.0 equiv.), 113 mL of water and 225 mL of ethanol was added 5.18 g of 10% palladium on carbon. This mixture was hydrogenated in a Parr apparatus at 60 psi for sixteen hours. The mixture was filtered through Celite. The ethanol was removed in vacuo and the pH adjusted to 9, at which point the product had precipitated. The precipitate was isolated by filtration and dried in vacuo to yield 18.0 g (80%) of N-[(4-aminophenyl)methyl]methanesulfonamide.

### N-[(4-Hydrazinylphenyl)methyl]methanesulfonamide hydrochloride

To a suspension of 18.0 g (89.9 mmol, 1.0 equiv.) of N-[(4-aminophenyl)methyl]methanesulfonamide in 85 ml of water and 175 mL of concentrated aqueous hydrochloric acid at 0° C. was added a solution of 6.21 g (89.9 mmol, 1.0 equiv.) of sodium nitrite in 85 mL of water. The reaction mixture was stirred for fifteen minutes. Meanwhile a solution of 101.6 g of stannous chloride dihydrate in 175 mL of concentrated aqueous hydrochloric acid was prepared and cooled to −55° C. The solution of the diazonium salt was filtered into the stannous chloride solution. The reaction was maintained between −55° C. and −35° C. for one hour. The crystalline material was collected by filtration and dried under high vacuum at 65° C. for 16 hours to yield 23.9 g (>100%) of crude N-[(4-hydrazinylphenyl)methyl]methanesulfonamide hydrochloride.

### N-[[4-[2-(5-Hydroxypentylidene)hydrazinyl]phenyl]methyl]methanesulfonamide

To a solution of 23.9 g of crude N-[(4-hydrazinylphenyl)methyl]methanesulfonamide hydrochloride in 175 mL of water and 300 mL of ethanol was added sufficient sodium acetate to bring the pH to 4. To the resulting suspension was added 10.4 mL (114 mmol, 1.3 equiv.) of 3,4-dihydro-2H-pyran. The mixture was then stirred for twenty hours at room temperature. The ethanol was removed in vacuo. The pH was raised to 10 by adding solid potassium carbonate and the mixture was extracted three times with 50 mL portions of methylene chloride. The combined organic layers were washed with 25 mL of saturated aqueous sodium chloride, dried over magnesium sulfate and filtered to give an oil. The oil was purified by chromatography on silica gel using 5% methanol in methylene chloride containing 0.5% concentrated aqueous ammonium hydroxide to yield 11.1 g (39%, two steps) of pure N-[[4-[2-(5-hydroxypentylidene)hydrazinyl]phenyl]methyl]methanesulfonamide.

### N-[[3-(3-Hydroxypropyl)-1H-indol-5-yl]methyl]methanesulfonamide

To a solution of 25.2 g (185 mmol, 5.0 equiv.) of zinc chloride in 900 mL of anhydrous 1,2-dimethoxyethane under nitrogen was added a solution of 11.1 g (37.0 mmol, 1.0 mmol) of N-[[4-[2-(5-hydroxypentylidene)hydrazinyl]phenyl]methyl]methanesulfonamide. The solution was refluxed for forty-eight hours then cooled to room temperature. The reaction mixture was filtered and concentrated in vacuo. The residue was dissolved in 500 mL of ethyl acetate and washed with 100 mL portions of water and saturated aqueous sodium chloride. The organic layer was dried over magnesium sulfate, filtered and concentrated in vacuo to yield 11.0 g of an oil. This was purified by chromatography on silica gel using gradient elution from 5% methanol in methylene chloride containing 0.5% concentrated aqueous ammonium hydroxide to 10% methanol in methylene chloride containing 1% concentrated aqueous ammonium hydroxide to yield 6.97 g (67%) of pure N-[[3-(3-hydroxypropyl)-1H-indol-5-yl]methyl]methanesulfonamide.

Substitution of an acyl halide for the alkylsulfonyl chloride in step 1 of the above sequence provide the acyl analog of the sulfonyl product.

### Compounds of Type:

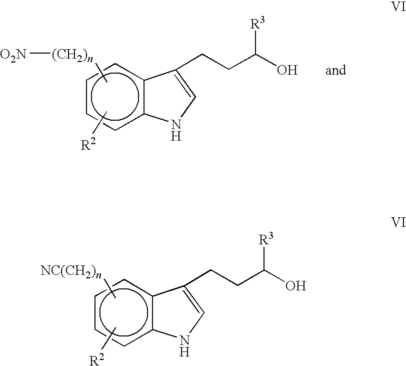

### Example 8

### (5-Cyano-1H-indol-3-yl)propanol

### 5-Cyano-3-(dimethylamino))methyl-1H-indole

To a solution of 14.0 g (98.5 mmol, 1.0 equiv.) of 5-cyanoindole in 35 mL of ethanol was added 8.7 mL (108 mmol, 1.1 equiv.) of 40% aqueous formaldehyde and 12.2 mL (108 mmol, 1.1 equiv) of 40% aqueous dimethylamine. The flask was fitted with a reflux condenser topped with a cold finger condenser at −78° C. The solution was heated to reflux for eight hours at which time TLC indicated absence of starting material. The solution was poured into 350 mL of chloroform and washed once with 50 mL of 10% aqueous sodium carbonate. The organic layer was separated, dried over anhydrous sodium sulfate, filtered and concentrated to yield 19.6 g (100%) of 5-cyano-3-(dimethylamino)methyl-1H-indole, pure by ¹H NMR analysis.

### N,N,N-trimethyl-1-(5-cyano-1H-indol-3-yl)methanaminium iodide

To a solution of 2.7 g (13.6 mmol, 1.0 equiv.) of 5-cyano-3-(dimethylamino)methyl-1H-indole in 125 mL of THF at 0° C. was added 1.3 mL (20.3 mmol, 1.5 equiv.) of methyl iodide dropwise. After five minutes the solution was allowed to warm to room temperature and stirred for one hour. The resulting precipitate was filtered and dried in vacuo to yield 4.4 g (96%) of N,N,N-trimethyl-1-(5-cyano-1H-indol-3-yl)methanaminium iodide, pure by ¹H NMR analysis.

### Methyl 2-methoxycarbonyl-3-(5-cyano-1H-indol-3-yl)propanoate

To a solution of 21.5 g (63.0 mmol, 1.0 equiv.) of N,N,N-trimethyl-1-(5-cyano-1H-indol-3-yl)methanaminium iodide in 300 mL of methanol was added 14.6 g (94.5 mmol, 1.5 equiv.) of sodiodimethylmalonate. The mixture was warmed to reflux for sixteen hours. The solution was cooled to room temperature and diluted with 1000 mL of chloroform. The organic layer was washed once with 250 mL of saturated aqueous sodium bicarbonate solution. The organic layer was separated, dried over anhydrous sodium sulfate, filtered and concentrated. The resulting residue was filtered through a plug of silica gel with 1:1 ethyl acetate:hexane to remove unreacted dimethyl malonate. This provided 18.95 g (100%) of methyl 2-methoxycarbonyl-3-(5-cyano-1H-indol-3-yl)propanoate

### Methyl 3-(5-cyano-1H-indol-3-yl)propanoate

To a solution of 1.14 g (3.98 mmol, 1.0 equiv.) of methyl 2-methoxycarbonyl-3-(5-cyano-1H-indol-3-yl)propanoate in 20 mL of pyridine was added 1.19 g (7.96 mmol, 2.0 equiv.) of sodium iodide. The mixture was heated to reflux for sixteen hours. The solution was cooled and diluted with 150 mL of chloroform. The organic layer was washed once with 25 mL of saturated aqueous sodium bicarbonate solution. The organic layer was separated, dried over anhydrous sodium sulfate, filtered and concentrated. The resulting residue was filtered through a plug of silica gel with 1:1 ethyl acetate:hexane to remove colored impurities to provide 0.734 g (81%) of methyl 3-(5-cyano-1H-indol-3-yl)propanoate.

### (5-Cyano-1H-indol-3-yl)propanol

To a suspension of 0.184 g (4.86 mmol, 2.0 equiv.) of lithium aluminum hydride in 10 mL of THF at 0° C. was added a solution of 0.555 g of methyl 3-(5-cyano-1H-indol-3-yl) propanoate dropwise. The resulting suspension was stirred at 0° C. for thirty minutes then quenched with 0.18 mL of water, 0.18 mL of 15% aqueous sodium hydroxide and 0.54 mL of water. The resulting suspension was filtered through Celite. The solution was concentrated and the residue filtered through a plug of silica gel with 1:1 ethyl acetate:hexane to yield 0.470 g (97%) of (5-cyano-1H-indol-3-yl)propanol.

### Example 9

### 5-Nitro-3-(3-hydroxypropyl)indole

### 5-(5-Nitroindol-3-ylmethyl)-2,2-dimethyl-1,3-dioxane-4,6-dione

An adaption of the procedure of Flaugh was used. Thus, a solution of 5-nitroindole (50.0 g, 0.31 mol), Meldrum's acid (46.0 g, 0.32 mol), 37% aqueous formaldehyde (26.0 mL, 0.32 mol) and proline (1.8 g, 0.016 mol) in 200 mL of acetonitrile was stirred at room temperature for 18 h. The resulting thick yellow slurry was filtered and the filtercake was washed with acetonitrile, then acetone and finally with ether. This material was dried in vacuo to give the title compound (80.0 g, 81%) as a bright yellow solid, m.p. 182° C. (dec). The mother liquor was concentrated and then diluted with H₂O, and the resulting solid was collected and washed and dried as before. This gave a second crop of the product (7.0 g) as a darker yellow solid. Total yield=87.0 g (89%).

### Ethyl 5-nitro-3-indolepropionate

To a solution of 5-(5-nitroindol-3-ylmethyl)-2,2-dimethyl-1,3-dioxane-4,6-dione (10.0 g, 0.031 mol) in a mixture of pyridine (80 mL) and absolute ethanol (20 mL) was added 0.1 g of copper powder and the mixture was refluxed under Ar for 2 h. After being stirred at room temperature for 66 h the mixture was refluxed for an additional 1 h. The cooled mixture was filtered and the filtrate was evaporated. The resulting residue was triturated with ether and a little CH₂Cl₂ to give the title compound (7.3 g, 89%) as a solid, m.p. 118°-121° C.

### 5-Nitro-3-(3-hydroxypropyl)indole

To a suspension of 95% LiAlH₄ (2.20 g, 0.058 mol) in 60 mL of dry THF was added a solution of ethyl 5-nitro-3-indolepropionate (7.30 g, 0.028 g mol) in 100 mL of dry THF, at 0° C. under Ar. After stirring for 20 min the mixture was quenched by cautiously adding 3 mL of H₂O. The resulting suspension was stirred for 10 min and then it was filtered and the filtercake was washed with additional THF. The filtrate was evaporated and the residue was taken up in ether, dried (Na₂SO₄) and evaporated, and the resulting solid was triturated with hexane to give the title compound (4.30 g, 70%) as a yellow solid, m.p. 107°-110° C.

### Example 10

### 1-[5-Acetyl-1H-indol-3-yl]-3-propanol

### 4-Amino-3-iodoacetophenone

To a solution containing 4-aminoacetophenone (4.05 g, 30 mmol) in glacial acetic acid (60 mL) and water (10 mL) was added dropwise a solution of iodine monochloride (4.97 g, 31 mmol) in acetic acid (15 mL). After the addition was complete, the reaction was heated at 90° C. for five min. then cooled to 23° C. and allowed to stand for one h. The excess IC1 was discharged by the addition of saturated sodium bisulfite (15 mL). The solvent was removed in vacuo and the residue dissolved in CH₂Cl₂, washed with saturated NaCl and finally with water. The organic phase was dried with Na₂SO₄, filtered, and concentrated in vacuo. Silica gel chromatography (Hexane:ethyl acetate gradient; 10-50% EtOAc) afforded 4-amino-3-iodoacetophenone (6.15 g, 82.3%).

### 5-Acetyl-3-[3-(t-butyldimethylsiloxy)propyl]-2-trimethylsilyl-1H-indole

To a solution of 4-amino-3-iodoacetophenone (6.15 g, 25 mmol) in 1,2-dimethoxyethane (250 mL) was added saturated Na₂CO₃ (20 mL), 1-trimethylsilyl-5-t-butyldimethylsiloxypent-1-yne (13.36 g, 49 mmol), and Pd(PPh₃)₄ (2.85 g, 2 mmol). After heating the mixture at reflux for 48 h the solvent was removed in vacuo. The residue was dissolved in ethyl acetate and washed with saturated NaCl and next with water. The organic phase was dried over MgSO₄, filtered, and concentrated in vacuo. Silica gel chromatography (Hexane:ethyl acetate gradient; 10-50% EtOAc) and crystallization from CH₂Cl₂:hexanes afforded 5-acetyl-3-[3-(t-butyldimethylsiloxy)propyl]-2-trimethylsilyl-1H-indole (5.76 g, 58%).

### 1-[5-Acetyl-1H-indol-3-yl]-3-propanol

To a solution of 5-acetyl-3-[3-(t-butyldimethylsiloxy)propyl]-2-trimethylsily 1-1H-indole (1.8 g, 5 mmol) in acetonitrile (100 mL) was added a 50% HF solution (4 mL). After stirring for 24 h at 23° C., the reaction was made basic (pH 10) by the addition of 50% NaOH solution and concentrated in vacuo. The residue was dissolved in ethyl acetate and washed with saturated NaCl and next with water. The organic phase was dried over MgSO₄, filtered, and concentrated in vacuo. Silica gel chromatography (CH₂Cl₂:MeOH:NH₄ OH; 95:5:0.5) of the concentrate afforded 1-[5-acetyl-1H-indol-b3-yl]-3-propanol (0.91 g, 94%).

### Example 11

### 5-Fluoro-3-(3-hydroxypropyl)indole

### 5-Fluoroindole-3-propionic acid

A modification of a procedure reported by Johnson (H. E. Johnson and D. G. Crosby, J. Org. Chem., 25, 569 (1969)) for the preparation of indole-3-propionic acid was used. Thus, a solution of 5-fluoroindole (1.35 g, 0.010 mol) in 10 mL of acetic acid containing acrylic acid (1.5 mL, 0.022 mol) and acetic anhydride (1.9 mL, 0.02 mol) was heated (oil bath) at 90° C. under Ar for 5 days. The volatiles were then removed in vacuo and the residue was taken up in 3N NaOH. Insoluble material was removed by filtration and the filtrate was acidified with conc. HCl and then extracted with CH₂Cl₂. The organic extract was dried (Na₂ SO₄) and evaporated to give the product (1.191 g, 57%) as a solid which was used without further purification: IR (neat) 3420, 1710 cm⁻¹; ¹Hnmr (200 MHz, CDCl₃) δ7.94 (br s, 1H), 7.28-7.18 (m, 3H), 7.05 (d, J=2.5 Hz, 1H), 6.93 (dt, J=9.0, 2.6 Hz, 1H), 3.05 (t, J=7.5 Hz, 2H), 2.73 (t, J=7.6 Hz, 2H).

By appropriate modification of the general method, other Formula II compounds are readily obtainable.

### 5-Fluoro-3-(3-hydroxypropyl)indole

To a suspension of LiAlH₄ (433 mg, 11.4 mmol) in 20 mL of dry tetrahydrofuran at 5°-10° C. under Ar was added a solution of 5-fluoroindole-3-propionic acid (1.179 g, 5.7 mmol) in 5 mL of tetrahydrofuran. After 10 min the cooling bath was removed and the mixture was stirred at room temperature for 30 min and finally it was heated to reflux for 30 min. The resulting gummy mixture was allowed to cool to room temperature and then the reaction was quenched by the sequential addition of 0.5 mL of H₂O, 0.5 mL of 15% NaOH solution and finally 1.5 mL of H₂O. The mixture was then diluted with ethyl acetate, dried (MgSO₄) and evaporated to give a yellow-green oil. Flash chromatography (SiO₂/CH₂Cl₂-ethyl acetate=2:1) afforded the product (918 mg, 83%) as an oil: IR (neat) 3420, 1583 cm⁻¹; Hnmr (200 MHz, CDCl₃) δ7.94 (br s, 1H), 7.28-7.20 (m, 2H), 7.03 (d, J=2.4 Hz, 1H), 6.92 (dt, J=9.1, 2.5 Hz, 1H), 3.71 (t, J=6.4 Hz, 2H), 2.80 (t, J=7.5 Hz, 2H), 2.02-1.88 (m, 2H), 1.33 (br s, 1H).

### Example 12

### Ethyl 3-[3-hydroxyprop-1-1H-indole-5-carboxylate

### Ethyl 3-[3-[[(1,1-dimethylethyl)dimethylsilyl]oxy]prop-1-yl]-2-trimethylsilyl-1H-indole-5-carboxylate

A mixture of ethyl 4-amino-3-iodobenzoate {Hirschfeld et al. J. Med Chem. 1992, 35, 2231-2238.} (7.80 g, 26.8 mmol), [5-[[(1,1-dimethylethyl)dimethylsilyl]oxy]-1-pentynyl]trimethylsilane (9.41 g, 34.8 mmol), tetra-n-butylammonium chloride (7.44 g, 26.8 mmol), sodium carbonate (14.20 g, 0.134 mol), triphenylphosphine (0.35 g, 1.3 mmol), and palladium (II) acetate (0.30 g, 1.3 mmol) in 200 mL of DMF, was heated at 98° C. under N₂ for 2 h. The solution was cooled, and the bulk of the DMF was removed in vacuo. The mixture was diluted with ethyl acetate, and the organic solution was washed with aqueous NaHCO₃, dried (brine, MgSO₄), filtered, and concentrated in vacuo. Silica gel chromatography (10:1 hexanes-EtOAc) of the concentrate yielded the title compound (7.49 g, 65%) as a crystalline solid. Recrystallization from hexanes provided an analytical sample as a white crystalline solid: mp 115°-116° C. Anal. Calcd for C₂3H₃9N₁O₃Si₂: C, 63.69; H, 9.06; N, 3.23. Found: C, 63.54; H, 9.11; N, 3.17.

### Ethyl 3-[3-hydroxy-prop-1-yl]-1H-indole-5-carboxylate

A solution of ethyl 3-[3-[(1,1-dimethylethyl)dimethylsilyloxy]prop-1-yl]-2-trimethylsilyl-1H-indole-5-carboxylate (5.0 g, 11.5 mmol), and aqueous 48% HF (1.9 g, 46.0 mmol) in 140 mL of MeCN was stirred at 23° C. for 3.5 h. The solution was quenched with 10% Na₂CO₃, and the organic material was extracted into ethyl acetate. The organic solution was dried (brine, MgSO₄), filtered, and concentrated in vacuo. Silica gel chromatography (50-100% EtOAc-hexanes gradient) of the concentrate yielded the title compound (2.79 g, 98%) as a white crystalline solid: mp 111°-112° C. Anal. Calcd for C₁4H₁7N₁O₃: C, 68.00; H, 6.93; N, 5.66. Found: C, 67.94; H, 6.78; N, 5.65.

### Example 13

### 3-(3-Hydroxydroyl)-1H-indole-5-carboxylic acid

To a solution of methyl 3-(3-hydroxypropyl)-1H-indole-5-carboxylate (5.0 g, 21.46 mmol) in 50 mL ethanol was added 30 mL of 15% potassium hydroxide in ethanol. The mixture was heated to reflux for 1 h. The mixture was cooled to 0° C. and acidified with concentrated hydrochloric acid to pH 6. The ethanol was removed in vacuo. The residue was diluted with 250 mL of ethyl acetate and washed with 50 mL of water. The organic layer was dried over anhydrous sodium sulfate, filtered and concentrated in vacuo to yield 3-(3-hydroxypropyl)-1H-indole-5-carboxylic acid (4.30 g, 92%).

### Example 14

### 3-(3-Hydroxypropyl)-N-(phenylmethyl)-1H-indole-5-carboxamide

To a mixture of 3-(3-hydroxypropyl)-1H-indole-5-carboxylic acid (4.30 g, 19.63 mmol), triethylamine (3.97 g, 39.26 mmol) and benzylamine (2.10 g, 19.63 mmol) in 150 mL acetonitrile was added 2-chloro-1-methylpyridinium iodide (6.04 g, 23.68 mmol). The mixture was heated to reflux for 16 h. The solution was cooled to room temperature, 70 mL of 0.5N hydrochloric acid added, and the acetonitrile removed in vacuo. The residue was extracted with 250 mL of ethyl acetate. The organic layer was separated and the aqueous layer reextracted with three 50 mL portions of ethyl acetate. The combined organic layers were dried over anhydrous sodium sulfate, filtered and concentrated in vacuo. Silica gel chromatography (95:5:0.5 CH₂Cl₂-MeOH—NH₄OH) of the concentrate yielded 3-(3-hydroxypropyl)-N-(phenylmethyl)-1H-indole-5-carboxamide (2.26 g, 37%).

### Compounds of Formula V

The conversion of Formula VI compounds to compounds of Formula V is accomplished by standard synthetic reactions in which an alkanol moiety is converted to an alkyl-[leaving group] moiety. The following examples are intended to be demonstrative but not limiting.

### Example 15

### 3-(5-Ethanesulfonylamino-1H-indol-3-yl)propyl methanesulfonate

To a solution of 3-(5-ethanesulfonylamino-1H-indol-3-yl)propanol (0.59 g, 2.1 mmol) in 20 mL of THF at 0° C. was added triethylamine (0.58 mL, 4.2 mmol) followed by the dropwise addition of methanesulfonyl chloride (0.24 mL, 3.1 mmol). The reaction was stirred for 30 minutes at 0° C. The reaction was poured into a mixture of saturated NaHCO₃ and ethyl acetate. The organic phase was separated and the aqueous phase reextracted with three portions of ethyl acetate. The combined organic layers were washed with a saturated NaCl solution, dried over anhydrous K₂CO₃, filtered, and concentrated in vacuo to yield 3-(5-ethanesulfonylamino-1H-indol-3-yl)propyl methanesulfonate (0.75 g, >99%) which was used without further purification.

### Example 16

### 3-[5-[(Phenylmethoxycarbonyl)amine]-1H-indol-3-yl]propyl-4-methylbenzenesulfonate

To a solution of alcohol (0.31 g, 0.96 mmol), triethylamine (0.15 g, 1.4 mmol), and 4-dimethylaminopyridine (DMAP) (0.01 g) in CH₂Cl₂ (10 mL) at 0° C. was added TsCl (0.27 g, 0.96 mmol). The reaction was allowed to warm to 23° C. and stand 16 h. At the end of this time, additional TsCl (0.068 g) and triethylamine (0.038 g) were added and allowed to react for 3 h. The reaction was treated with ice/water and the organic phase was separated, extracted with saturated NaCl solution, dried with K₂CO₃, filtered, and concentrated in vacuo. Silica gel chromatography (70:30 hexane:ethyl acetate) of the concentrate afforded 3-[5-[(phenylmethoxycarbonyl)amine]-1H-indol-3-yl]propyl 4-methylbenzenesulfonate (0.39 g, 85%) whose structure was confirmed by NMR and MS analysis.

### Example 17

### 3-[5-[[(Methylamino)sulfonyl]methyl]-1H-indol-3-yl]-propylmethanesulfonate

To a solution of 5.16 g (18.3 mmol, 1.0 equiv.) of 3-(3-hydroxypropyl)-N-methyl-1H-indole-5-methanesulfonamide and 3.84 mL (27.4 mmol, 1.5 equiv.) of triethylamine in 100 mL of anhydrous acetonitrile and 100 mL of methylene chloride at 0° C. was added 1.77 mL (22.8 mmol, 1.25 equiv.) of methanesulfonyl chloride dropwise. The reaction was stirred for thirty minutes at 0° C. then diluted with 250 mL of ethyl acetate. The organic layer was washed with 50 mL portions of saturated aqueous sodium bicarbonate and saturated aqueous sodium chloride. The organic layer was dried over anhydrous magnesium sulfate, filtered and concentrated in vacuo to yield an oil which was used directly in the next step.

### Example 18

### 5-Nitro-3-(3-bromopropyl)indole

To a solution of triphenylphosphine (6.70 g, 0.025 mol) in 80 mL of acetonitrile was added a solution of 5-nitro-3-(3-hydroxypropyl)indole (4.30 g, 0.020 mol) in 75 mL of acetonitrile, followed by a solution of CBr₄ (9.00 g, 0.027 mol) in 25 mL of acetonitrile, at 0° C. under Ar. The mixture was stirred at room temperature for 3 h and then it was evaporated and the residue was chromatographed (SiO₂/EtOAc-hexane, 1:9 then 1:4) to give the title compound (4.60 g, 84%) as a solid, m.p. 92°-95° C.

### Example 19

### 5-Fluoro-3-(-p-toluenesulfonyloxypropyl)indole

To a solution of 5-fluoro-3-(3-hydroxypropyl)indole (917 mg, 4.75 mmol) in 20 mL of CH₂Cl₂ at 0° C. under Ar was added triethylamine (728 .mu.L, 5.23 mmol), followed by a solution of p-toluenesulfonyl chloride (994 mg, 5.23 mmol) in 5 mL of CH₂Cl₂ and then a catalytic amount of 4-dimethylaminopyridine (59 mg, 0.48 mmol). The reaction mixture was stirred at 0° C. for 30 min and then at room temperature for 1½ h. Evaporation of the mixture followed by chromatography (SiO₂/CH₂Cl₂) of the residue gave a gum. The gum was dissolved in ether and then the solution was diluted with hexane until an oil separated. Addition of a small amount of CH₂Cl₂ led to dissolution of the oil and crystallization of the product. Storage at −20° C. and then filtration and drying of the residue in vacuo gave the product (1.378 g, 84%) as fluffy white needles m.p. 99° C.; IR (CH₂Cl₂) 3470, 1360, 1178 cm⁻¹; ¹Hnmr (200 MHz, CDCl₃) δ7.90 (br s, 1H), 7.61 (d, J=8.4 Hz, 2H), 7.30 (d, J=8.4 Hz, 2H), 7.27-7.20 (m, 1H) 7.08 (dd J=9.62.6 Hz, 1H) 6.96-6.94 (m, 1H), 6.88 (dd, J=9.0, 2.5 Hz, 1H), 4.06 (t, J=6.2 Hz, 2H), 2.74 (t, J=7.2 Hz, 2H), 2.42 (s, 3H), 1.99 (dq, J=7.2, 6.2 Hz, 2H).

### Example 20

### Ethyl 3-[3-iodopropy-1-yl]-1H-indole-5-carboxylate

A mixture of ethyl 3-[3-hydroxy-prop-1-yl]-1H-inodole-5-carboxylate (2.64 g, 10.7 mmol), triethylamine (1.52 g, 15 mmol), and methanesulfonyl chloride (1.83 g, 16 mmol) in 50 mL of acetonitrile was stirred at 0° C. for 1 h. The acetonitrile was removed in vacuo and the organic material was diluted with ethyl acetate. The organic solution was washed with aqueous Na₂CO₃, dried (brine, MgSO₄), filtered, and concentrated in vacuo. The concentrate was dissolved in 100 mL of acetonitrile and powdered KI (3.55 g, 21.4 mmol) added. The solution was heated at reflux for 1.5 h, then cooled, poured into water and extracted into EtOAc. The combined organic extracts were dried (brine, MgSO₄), filtered, and concentrated in vacuo. Silica gel chromatography (5:1 hexanes-EtOAc) of the concentrate yielded the title compound (3.50 g, 92%) as a white crystalline solid: mp 81° C. Anal. Calcd for C₁4H₁6I₁N₁O₂: C, 47.08; H, 4.51; N, 3.92. Found: C, 47.12; H, 4.44; N, 3.89.

### Compounds of Formula III

### Example 21

### 1-(5-Methoxy-4-pyrimidinyl)piperazine

### Method 1

To a solution of piperazine (38.40 g, 0.45 mole) in CH₃CN (225 mL) was added dropwise a CH₃CN (100 mL) solution containing 4-chloro-5-methoxypyrimidine (6.45 g, 0.04 mole) while under nitrogen atmosphere. After the addition was complete the reaction was heated at 60° C. for 0.75 h. The reaction was concentrated under reduced pressure and the residue dissolved in CH₂Cl₂ and extracted with 5% NaHCO₃ and H₂O. The organic phase was dried with K₂CO₃, filtered, and concentrated under reduced pressure. Silica gel chromatography (CH₂Cl₂:MeOH:NH₄ OH; 92:8:0.8) of the concentrate afforded II (7.63 g, 88.1%). Treatment of the base (1.0 g) with ethanolic HCl and crystallization from EtOH/i-PrOH yielded the hydrochloride salt of II (0.50 g, 39.1%, m.p. 2070-211°).

### 1-(5-Methoxy-4-pyrimidinyl)piperazine

### Method 2

### 4,6-Dihydroxy-5-methoxypyrimidine

A modified procedure of Bretschneider, Richter, and Klotzer, Monatsh. Chem. 96(6), 1661-76 (1965), was used. Absolute methanol (1.0 L) was added with ice bath cooling to sodium methoxide (175 g, 3.24 moles) in a 3 L round bottom flask. When the mixture had cooled to less than 20° C., dimethyl methoxymalonate (162.14 g, 1.00 mole) was added, and then solid formamidine acetate (104.11 g, 1.00 mole) was added. The mixture was stirred i the ice bath for 30 minutes, and then refluxed for 1 hour. The mixture was cooled in a cold water bath and then concentrated HCl (about 350 mL) was added until the mixture was strongly acidic on pH test paper. The precipitate was filtered, suspended in cold water (about 400 mL), and then filtered again. The white powder was dried in vacuo (125.84 g, 88.7%), and carried on without further purification.

### 4,6-Dichloro-5-methoxypyrimidine

A modified procedure of Bretschneider, Richter, and Klotze, Monatsh. Chem. 96(6), 1661-76 (1965), was used. A mixture of 4,6-dihydroxy-5-methoxy-pyrimidine (125.84 g, 0.887 mole), POCl₃ (700 mL), and N,N-diethylaniline (50 mL) was refluxed for 3 hours to give a brown solution. The solution was cooled and then the excess POCl₃ was removed in vacuo. Hexane (about 300 mL) was added to the residue and the mixture was refluxed with stirring. The hot hexane layer was decanted into a beaker, and residue treated two more times with hot hexane. The hexane extracts (total volume about 1 L) were concentrated in vacuo to give the crude product as a white solid (116.5 g, 73.3%). This material was recrystallized from pet ether to give colorless needles (92.0 g+16.51 g second crop, 93.1% total recovery).

### 4-Chloro-5-methoxy-4-(1-piperazinyl)pyrimidine

Piperazine (30 g) was dissolved in water (150 mL) and then solid 4,6-Dichloro-5-methoxypyrimidine (10.00 g, 55.8 mmole) was added. The mixture was vigorously stirred for 2 h at room temperature during which the 4,6-dichloro-5-methoxypyrimidine dissolved. The product was extracted from the aqueous reaction mixture with methylene chloride (yield 12.67 g, 99.2%). A sample (5 g) of the crude product was chromatographed on silica gel using a gradient of 20-40% methanol/ethyl acetate as the eluent. The product was then dissolved in acetonitrile and concentrated HCl added to give the salt as a white powder which was dried in vacuo to give the analytical sample (4.0 g, m.p.: 169°-173° C. bubbles).

Anal. Calcd for C₉H₁3N₄OCl1.5HCl.0.2H₂O,

C, 37.67; H, 5.24; N, 19.53 H₂O; 1.26.

Found: C, 37.63; H, 4.99; N, 1946H₂O; 1.47.

### 1-(5-Methoxy-4-pyrimidinyl)piperazine

Piperazine (20 g) was dissolved in water (100 mL) in a Parr bottle and then solid 4,6-dichloro-5-methoxypyrimidine (5.00 g, 27.9 mmole) was added. The mixture was vigorously stirred for 2 h at room temperature during which the 4,6-dichloro-5-methoxypyrimidine dissolved. The stirring bar was removed, catalyst (10% Pd/C, 1.0 g) was added to the turbid solution, and the mixture was then hydrogenated (60 psi, 3 h) at room temperature. The catalyst was filtered off and the filtrate extracted 3 times with CH₂Cl₂. The CH₂Cl₂ extracts were dried over Na₂SO₄ and concentrated in vacuo to give a clear oil which solidified upon standing (3.34 g, 61.7%). This crude product was Kugelrohr distilled (yield 3.24 g), dissolved in acetonitrile, and concentrated HCl was added to precipitate the product as a white powder which was dried in vacuo (4.32 g, 94.0% from crude product, m.p. 2190-221.5° C.).

### Example 22

### 1-(5-Methoxy-4-pyrimidinyl)-2-methylpiperazine

### Method 1

### 1-(t-Butoxycarbonyl)-3-methylpiperazine

To a cold (−5° C.) solution of 2-methylpiperazine (5.00 g, 0.05 mole) in 200 mL of CH₂Cl₂ under Ar was added a solution of di-t-butyl dicarbonate (10.9 g, 0.05 mole) in 100 mL of CH₂Cl₂ over 1 h. The resulting mixture was stirred at −5° C. for 1 h and then at r.t. for 2 h. The solution was then washed (H₂O), dried (Na₂SO₄) and evaporated to give an oil which was chromatographed (SiO₂/ethyl acetate then ethyl acetate-MeOH—NH₄OH 10:1:0.1) to give the product (4.30 g, 43%) as an oil. This material was used without further purification: ¹H nmr (200 MHz, CDCl₃) δ4.15-3.75 (br s, 2H), 3.0-2.6 (m, 4H), 2.47-2.35 (m, 1H), 1.48 (s, 9H), 1.08 (d, J=6.7 Hz, 3H).

### 1-(t-Butoxycarbonyl)-4-(5-methoxy-4-pyrimidyl)-3-methylpiperazine

A mixture of 1-(t-butoxycarbonyl)-3-methylpiperazine (2.0 g, 0.01 mole), 4-chloro-5-methoxypyrimidine (1.5 g, 0.01 mole) and diisopropylethylamine (2.6 mL, 0.015 mole) in 25 mL of dry acetonitrile was heated to reflux under Ar for 60 h. The resulting solution was diluted with ether and then washed (H₂O, brine), dried (Na₂SO₄) and evaporated to give a gum. This gum was triturated with hexane (.times.3) and the supernatant was evaporated to give a gum. Flash chromatography (SiO₂/ethyl acetate-hexane=1:1, then ethyl acetate) of this material gave first 4-chloro-5-methoxypyrimidine (0.4 g, 27%) and then the desired product (1.2 g, 30%) as a light pink solid: m.p. 70°-72° C.; IR (KBr) 1690, 1575 cm⁻¹; ¹H nmr (200 MHz, CDCl₃) δ8.33 (s, 1H), 7.90 (s, 1H), 4.79 (br s, 1H), 4.4-3.8 (m, 3H), 3.86 (s, 3H), 3.35-2.90 (m, 3H), 1.48 (s, 9H), 1.21 (d, J=6.7 Hz, 3H).

### 1-(5-Methoxy-4-pyrimidinyl)-2-methylpiperazine

A solution of 1-(t-butoxycarbonyl)-4-(5-methoxy-4-pyrimidinyl)-3-methylpiperazine (1.70 g, 4.2 mmol) and trifluoroacetic acid (5 mL) in 50 mL of CH₂Cl₂ was stirred at r.t. under Ar for 18 h. The solution was evaporated, the residue was taken up in water and the mixture was basified (pH8) with 15% aqueous NaOH. The resulting (pH 8) mixture was extracted with ethyl acetate and the organic phase was washed (H₂O, brine), dried (Na₂SO₄) and evaporated to give a semi-solid. This material was taken up in ether, filtered to remove insoluble material and the solution was evaporated to give the product (0.80 g, 92%) as an oil. It was used without further purification: IR (neat) 3300, 1580, 1540 cm⁻¹; ¹H nmr (200 MHz, CDCl₃) δ8.32 (s, 1H), 7.87 (s, 1H), 4.83-4.68 (m, 1H), 4.26-4.19 (m, 1H), 3.85 (s, 3H), 3.26-3.17 (m, 1H), 3.12-3.01 (m, 2H), 2.94-2.80 (m, 2H), 1.29 (d, J=6.8 Hz, 3H).

### 4-(5-Methoxy-4-pyrimidinyl)-2-methylpiperazine

### Method 2

A solution of 2-methylpiperazine (20 g) in water (100 mL) was reacted with solid 4,6-dichloro-5-methoxypyrimidine (5.00 g, 27.9 mmole) in a procedure similar to that given for Method 2 of Example 14. After hydrogenation and filtration of the catalyst, the product was extracted from the filtrate with CH₂Cl₂. The extracts were concentrated in vacuo, and the residue was Kegelrohr distilled to give a clear oil (5.46 g, 99.8%). The oil was dissolved in acetonitrile and concentrated HCl added to form the salt which was recrystallized from i-PrOH and dried in vacuo to give the product as a White powder (4.02 g, m.p. 185°-188° C.).

### Example 23

### 1-(5-Ethoxy-4-pyrimidinyl)piperazine

### 5-Ethoxypyrimidine

Sodium (2.89 g. 125.8 mmol) was dissolved in ethanol (110 mL) and 5-bromopyrimidine (10.0 g, 62.9 mmol) added. The reaction was heated at 120° in an autoclave for 17 h and then allowed to stand at 230 for 60 h. The ethanol was removed under reduced pressure and water (5 mL) added to the concentrate. The aqueous phase was extracted with CH₂Cl₂ (4.times.100 mL). The combined organic extracts were washed with saturated NaCl solution, dried with anhydrous K₂CO₃, filtered, and concentrated under reduced pressure. Silica gel chromatography (Hexane/EtOAc; 70:30) of the concentrate yielded 5-ethoxypyrimidine (2.00 g, 25.6%).

### 5-Ethoxypyrimidine N-oxide

To a solution of 5-ethoxypyrimidine (2.00 g, 16.1 mmol) in CH₂Cl₂ (100 mL) was added 3-chloroperoxybenzoic acid, 50-60% tech. grade, (6.13 g, 17.7 mmol) and the reaction stirred at 23° C. for 18 h. The reaction was extracted with water (2 mL) containing Na₂CO₃ (1.71 g, 16.1 mmol). The organic phase was dried with anhydrous K₂CO₃, filtered, and concentrated under reduced pressure to afford the N-oxide (1.75 g, 78%).

### 4-Chloro-5-ethoxypyrimidine

To a solution of triethylamine (1.90 g, 18.6 mmol) and phosphorous oxychloride (2.87 g, 18.6 mmol) in CHCl₃ (60 mL) was added 5-ethoxypyrimidine N-oxide (1.75 g, 12.5 mmol) portionwise. After the addition was complete, the reaction was heated at reflux for 3 h. Upon cooling the mixture to 0° C., CHCl₃ (60 mL) and water (10 mL) were added followed by portionwise addition of NaHCO₃ (3.15 g, 37.5 mmol). After the effervescence had ceased, the organic phase was separated, dried with MgSO₄, filtered, and concentrated under reduced pressure to afford the chloro product (1.98 g) which was used without further purification.

### 4-(Ethoxycarbonyl)-1-(5-ethoxy-4-pyrimidinyl)piperazine

A mixture of 4-chloro-5-ethoxypyrimidine (1.98 g, 12.5 mmol), ethyl 1-piperazinecarboxylate (5.93 g, 37.5 mmol) and micropulverized K₂CO₃ (5.18 g, 37.5 mmol) was heated at reflux in CH₃CN (75 mL) for 4 h. Upon cooling to 23° C., the solvent was removed under reduced pressure. The residue was dissolved in CH₂Cl₂ and washed with water (5 mL). The organic phase was dried with anhydrous K₂CO₃, filtered, and concentrated under reduced pressure. Silica gel chromatography (CH₂Cl₂/MeOH; 98:2) of the concentrate yielded product (2.29 g, 65%).

### 1-(5-Ethoxy-4-pyrimidinyl)piperazine

To a KOH solution (10N, 20 mL) was added 4-(ethoxycarbonyl)-1-(5-ethoxy-4-pyrimidinyl)piperazine (2.29 g, 8.18 mmol). The reaction was heated at reflux for 24 h after which time the water was removed under reduced pressure. The residue was dissolved in CH₂Cl₂, washed with saturated NaCl solution, dried with anhydrous K₂CO₃, filtered, and concentrated under reduced pressure. Silica gel chromatography (CH₂Cl₂/MeOH/NH₄ OH; 95:5:0.5) of the concentrate yielded III (0.71 g, 42%).

### Example 24

### 4-(5-Methoxy-4-pyrimidinyl)-2-methyl-piperazine

### Method 1

A mixture of 2-methylpiperazine (27.74 g. 0.28 mole) and 4-chloro-5-methoxypyrimidine (8.0 g, 0.06 mole) was heated in a Parr bomb at 100° C. for 1.5 h. The reaction mixture was dissolved in CH₂Cl₂ and extracted with 5% NaHCO₃ and H₂O. The organic phase was dried with K₂CO₃, filtered, and concentrated under reduced pressure. Silica gel chromatography (CH₂Cl₂:MeOH:NH₄ OH; 93:7:0.7) of the concentrate afforded product (9.02 g, 78.2%). Treatment of the base (1.0 g) with ethanolic HCl and crystallization from i-PrOH/EtoH yielded the hydrochloride salt (0.45 g, 32.1%, m.p. 191°-193° C.).

### 4-(5-Methoxy-4-pyrimidinyl)-2-methyl-piperazine

### Method 2

A solution of 2-methylpiperazine (20 g) in water (100 mL) was reacted with solid 4,6-dichloro-5-methoxypyrimidine (5.00 g, 27.9 mmol) in a procedure similar to that given for Method 2 of Example 21. After hydrogenation and filtration of the catalyst, the product was extracted from the filtrate with CH₂Cl₂. The extracts were concentrated in vacuo, and the residue was Kugelrohr distilled to give a clear oil (5.46 g, 99.8%). The oil was dissolved in acetonitrile and concentrated HCl added to form the salt which was recrystallized from i-PrOH and dried in vacuo to give the product as a white powder (4.02 g, m.p. 1.85°-188° C.

### Product Compounds of Formula I

### Example 25

### 1-[3-(5-Ethanesulfonylamino-1H-indol-3-yl)propyl]-4-(5-methoxy-4-pyrimidinyl)-3-methylpiperazine

To a solution of 3-(5-ethanesulfonylamino-1H-indol-3-yl)propyl methanesulfonate (0.75 g, 2.1 mmol) in 15 mL of acetonitrile was added diisopropylethylamine (0.54 g, 4.2 mmol), potassium iodide (0.05 g, 0.3 mmol), t-butylammonium hydrogensulfate (0.04 g, 0.1 mmol), and 4-(5-methoxy-4-pyrimidinyl)-3-methylpiperazine (0.87 g, 4.2 mmol). The reaction was heated to reflux for 20 h. The reaction was concentrated in vacuo and the residue treated with 5% NaHCO₃ and extracted with three portions of ethyl acetate. The combined organic layers were washed with a saturated NaCl solution, dried over anhydrous K₂CO₃, filtered, and concentrated in vacuo. Silica gel chromatography (98:2 CH₂Cl₂:MeOH) of the residue afforded 0.32 g (32%) of the desired material. The hydrochloride salt was prepared by addition of ethanolic HCl. The salt was recrystallized from ethanol to yield 1-[3-(5-ethanesulfonylamino-1H-indol-3-yl)propyl]-4-(5-methoxy-4-pyrimidin yl)-3-methylpiperazine hydrochloride hydrate (0.15 g, 39%), m.p. 212°-214° C. Analysis calcd. for C₂3H₃2N₆O₃S.2HCl.0.4H₂O.0.3 EtOH: C, 50.04; H, 6.52; N, 14.84, found: C, 50.12; H, 6.27; N, 14.90.

### Example 26

### 1-[3-[5-[Methyl(trifluoromethanesulfonyl)amino]-1H-indol-3-yl]propyl]-4-(5-methoxy-4-pyrimidinyl)piperazine

m.p. >230° C. was prepared in a similar manner starting from 5-trifluoromethanesulfonylamino-1H-indole. Analysis calcd. for C₂2H₂7F₃N₆O₃S.2.0HCl: C, 45.14; H, 5.00; N, 14.36; found: C, 44.75; H, 4.90; N, 14.42.

### Example 27

### 1-[3-[5-[[(Phenylmethoxy)carbonyl]amino]-1H-indol-3-yl]propyl]-4-(5-methoxy-4-pyrimidinyl)piperazine

A solution of 3-[5-[(phenylmethoxycarbonyl)amine]-1H-indol-3-yl]propyl 4-methylbenzenesulfonate (0.54 g, 1.13 mmol), 4-(5-methoxy-4-pyrimidinyl)piperazine (0.44 g, 2.26 mmol), diisopropylethylamine (0.29 g, 2.26 mmol), potassium iodide (0.19 g, 1.13 mmol), and t-butylammonium hydrogensulfate (0.02 g, 1.13 mmol) in CH₃CN (10 mL) was heated at reflux for 20 h. After concentrating the reaction in vacuo, the residue was treated with 5% NaHCO₃ and extracted with four portions of CH₂Cl₂. The combined organic phases were washed with saturated NaCl, dried with K₂CO₃, filtered, and concentrated in vacuo. Silica gel chromatography (CH₂Cl₂:MeOH 96:4) of the concentrate afforded 1-8 3-[5-[[(phenylmethoxy)carbonyl]amino]-1H-indol-3-yl]propyl]-4-(5-methoxy-4-pyrimidinyl)piperazine (0.44 g, 77%), mp 174°′175° C. Analysis calcd. for C₂8H₃2N₆O: C, 67.19; H, 6.45; N, 16.79, found: C, 67.05; H, 6.47; N, 16.88.

### Example 28

### 4-(5-Methoxy-4-pyrimidinyl)-1-[3-[5-[[(methylamino)sulfonyl]methyl]-1H-indol-3-yl]propyl]piperazine

To a solution of crude 3-]5-]](methylamino)sulfonyl]methyl]-1H-indol-3-yl]propyl methanesulfonate (18.3 mmol, 1.0 equiv.) in 225 mL of anhydrous acetonitrile was added 3.30 g (18.3 mmol, 1.0 equiv.) of potassium iodide, 3.81 mL (21.9 mmol, 1.2 equiv.) of N,N-diisopropylethylamine and 3.89 g (20.1 mmol, 1.1 equiv.) of 1-(5-methoxy-4-pyrimidinyl)piperazine. The mixture was heated to reflux for sixteen hours. The reaction mixture was concentrated in vacuo and the residue was taken up in 500 mL of chloroform. The organic layer was washed with 100 mL portions of 10% potassium carbonate and saturated aqueous sodium chloride. The organic layer was dried over anhydrous magnesium sulfate, filtered and concentrated in vacuo to yield an oil. The oil was purified by chromatography on silica gel using 5% methanol in methylene chloride containing 0.5% concentrated aqueous ammonium hydroxide followed by chromatography on silica gel using 20% methanol in ethyl acetate to obtain 4.83 g (58%) of 4-(5-methoxy-4-pyrimidinyl)-1-[3-[5-[[(methylamino)sulfonyl]methyl]-1H-indol-3-yl]propyl]piperazine. The hydrochloride salt was prepared by addition of 3N ethanolic HCl. Two recrystallizations from hot methanol gave 4.05 g (68%) of 4-(5-methoxy-4-pyrimidinyl)-1-[3-[5-[[(methylamino)sulfonyl]methyl]-1H-indol-3-yl]propyl]-piperazine hydrochloride, m.p. 170° C. (d). Elemental analysis calculated for C₂2H₃0N₆O₃S/3HCl: C, 46.52; H, 5.86; N, 14.80; found: C, 46.14; H, 6.22; N, 14.51.

### Example 29

### 4-(5-Methoxy-4-pyrimidinyl)-1-[3-5-[[(methylamino)sulfonyl]methyl]-1H-indol-3-yl]propyl]-3-methylpiperazine hydrochloride hydrate

In a similar manner the 4-(5-methoxy-4-pyrimidinyl)-3-methylpiperazine derivative was synthesized by substituting 1-(5-methoxy-4-pyrimidinyl)-2-methylpiperazine for 1-(5-methoxy-4-pyrimidinyl)piperazine in the alkylation step: C₂3H₃2N₆O₃S/2.8HCl/0.8 C₃H₈O, m.p. 148° C. (d). Elemental analysis calculated: C, 48.99; H, 6.67; N, 13.49; found: C, 48.98; H, 6.77; N, 13.27.

### Example 30

### 1-[3-[5-[[(Dimethylamino)sulfonyl]methyl]-1H-indol-3-yl]propyl]-4-(5-methoxy-4-pyrimidinyl)piperazine

To a solution of crude 3-[5-[[(dimethylamino)sulfonyl]methyl]-1H-indol-3-yl]propyl methanesulfonate (1.4 mmol, 1.0 equiv.) in 10 mL of anhydrous acetonitrile was added 0.225 g (1.50 mmol, 1.1 equiv.) of sodium iodide, 0.26 mL (1.50 mmol, 1.1 equiv.) of N,N-diisopropylethylamine and 0.291 g (1.50 mmol, 1.1 equiv.) of 1-(5-methoxy-4-pyrimidinyl)piperazine. The mixture was heated to reflux for sixteen hours. The reaction mixture was concentrated in vacuo and the residue was taken up in 50 mL of ethyl acetate. The organic layer was washed with a 10 mL portion of 10% potassium carbonate. The organic layer was dried over anhydrous sodium sulfate, filtered and concentrated in vacuo to yield an oil. The oil was purified by chromatography on silica gel using 5% methanol in methylene chloride containing 0.5% concentrated aqueous ammonium hydroxide to obtain 0.403 g (62%) of 1-[3-[5-[[(dimethylamino)sulfonyl]-methyl]-1H-indol-3-yl]propyl]-4-(5-methoxy-4-pyrimidinyl)piperazine. The hydrochloride salt was prepared by addition of 3N ethanolic HCl. Two recrystallizations from methanol gave 0.323 g (64%) of 1-[3-[5-[[(dimethyl-amino)sulfonyl]methyl]-1H-indol-3-yl]propyl]-4-(5-methoxy-4-pyrimidinyl)piperazine hydrochloride hydrate, m.p. 210° C. (d). Elemental analysis calculated for C₂3H₃2N₆O₃S/3HCl/0.5H₂O: C, 46.74; H, 6.14; N, 14.22; found: C, 46.82; H, 6.42; N, 14.09.

### Example 31

### 4-(5-Methoxy-4-pyrimidinyl)-1-[[5-[[(methylsulfonyl)amino]methyl]-1H-indol-3-yl]propyl]piperazine

To a solution of crude 3-[5-[[(methylsulfonyl)amino]methyl]-1H-indol-3-yl]propylmethanesulfonate (15.1 mmol, 1.0 equiv.) in 200 mL of anhydrous acetonitrile were added 2.51 g (15.1 mmol, 1.0 equiv.) of potassium iodide, 3.15 mL of N,N-diisopropylethylamine and 3.23 g of 1-(5-methoxy-4-pyrimidinyl)piperazine. The mixture was heated to reflux for 48 hours. The reaction mixture was cooled to room temperature, filtered and concentrated. The residue was dissolved in 250 mL of chloroform, washed with 50 mL portions of 10% aqueous potassium carbonate and saturated aqueous sodium chloride. The organic layer was dried over anhydrous magnesium sulfate, filtered and concentrated in vacuo to yield an oil. Chromatography on silica gel using 7.5% methanol in methylene chloride containing 0.75% concentrated aqueous ammonium hydroxide gave 3.93 g (57%) of 4-(5-methoxy-4-pyrimidinyl)-1-[[5-[[(methylsulfonyl)amino]methyl]-1H-indol-3-yl]propyl]piperazine. The hydrochloride was prepared by addition of 3N ethanolic HCl to an ethanolic solution of the base. Recrystallization from ethanol followed by drying in vacuo at 65° C. gave 0.268 g (53%) of the hydrochloride ethanolate: C₂2H₃0N₆O₃S/2.4HCl/0.4C₂H₆O, m.p. 204°-206° C. Elemental analysis calculated C, 48.51; H, 6.21; N, 14.89; found: C, 48.59; H, 6.21; N, 14.95.

### Example 32

### 4-(5-Methoxy-4-pyrimidinyl)-1-[[5-[[methyl(methylsulfonyl)amino]methyl]-1H-indol-3-yl]propyl]piperazine

To a solution of 1.93 g (4.21 mmol, 1.0 equiv.) of 4-(5-methoxy-4-pyrimidinyl)-1-[[5-[[(methylsulfonyl)amino]methyl]-1H-indol-3-yl]propyl]piperazine in 84 mL of anhydrous THF at −b 78° C. was added 1.9 mL (4.41 mmol, 1.05 equiv.) of a 2.32M solution of nBuLi in hexane dropwise. A precipitate formed during the addition. After the addition was complete the reaction mixture was allowed to stir for forty minutes at −78° C. The reaction was then placed in an ice bath and 0.275 mL (4.41 mmol, 1.05 equiv.) of methyl iodide added dropwise neat. The reaction was allowed to warm slowly in the ice bath to room temperature over sixteen hours. The reaction mixture was diluted with 250 mL of ethyl acetate and washed with 50 mL portions of 10% aqueous potassium carbonate and saturated aqueous sodium chloride. The organic layer was dried over anhydrous magnesium sulfate, filtered and concentrated in vacuo to yield an oil. The oil was chromatographed with 5% methanol in methylene chloride containing 0.5% concentrated aqueous ammonium hydroxide to yield 1.1 g of a solid. The solid was recrystallized to yield 0.760 g (38%) of pure 4-(5-methoxy-4-pyrimidinyl)-1-[[5-[[methyl-(methylsulfonyl)amino]methyl]-1H-indol-3-yl]propyl]piperazine, C₂2H₃0N₆O₃S, m.p. 164°-165° C. Elemental analysis calculated: C, 58.45; H, 6.82; N, 17.78; found: C, 58.12; H, 6.75; N, 17.56.

### Example 33

### 1-[[5-[[Ethyl(methylsulfonyl)amino]methyl]-1H-indol-3-yl]propyl]-4-(5-methoxy-4-pyrimidinyl)piperazine

In a similar manner to Example 32 the 1-[[5-[[ethyl(methylsulfonyl)amino]methyl]-1H-indol-3-yl]propyl]-4-(5-methoxy-4-pyrimidinyl)piperazine was synthesized by utilizing ethyl iodide in the alkylation step: C₂3H₃2N₆O₃S/3.7HCl, m.p. 197° C. (d). Elemental analysis calculated: C, 45.47; H, 5.92; N, 13.83; found: C, 45.32; H, 6.10; N, 13.73.

### Example 34

### 4-(5-Methoxy-4-pyrimidinyl)-1-[[5-[phenylmethyl (methylsulfonyl)amino 1-methyl]-1H-indol-3-yl]propyl]piperazine

In a similar manner to Example 32 4-(5-methoxy-4-pyrimidinyl)-1-[[5-[[phenylmethyl(methylsulfonyl)amino]-methyl]-1H-indol-3-yl]propyl]piperazine was synthesized by utilizing benzyl bromide in the alkylation step: C₂9H₃6N₆O₃S/1.1HCl, m.p. 203°-204° C. Elemental analysis calculated: C, 59.16; H, 6.35; N, 14.27; found: C, 59.04; H, 6.38; N, 14.11.

### Example 35

### 1-[[5-[[(Ethylsulfonyl amino]methyl]-1H-indol-3-yl]-propyl]-4-(5-methoxy-4-pyrimidinyl)piperazine

In a similar manner to Example 32 1-[[5-[[(ethylsulfonyl)amino]methyl]-1H-indol-3-yl]propyl]-4-(5-methoxy-4-pyrimidinyl)piperazine was synthesized by utilizing ethanesulfonyl chloride in the sulfonylation step: C₂3H₃2N₆O₃S/2.1C₄H₄O₄/0.6H₂O, m.p. 136° C. (d). Elemental analysis calculated: C, 51.70; H, 5.73; N, 11.38; found: C, 51.32; H, 5.70; N, 11.54.

### Example 36

### 1-[[5-[[(Methylsulfonyl)amino[methyl]-1H-indol-3-yl]propyl]-4-(5-methoxy-4-pyrimidinyl)-3-methylpiperazine

In a similar manner to Example 32 1-[[5-[[(methyl-sulfonyl)amino]methyl]-1H-indol-3-yl]propyl]-4-(5-methoxy-4-pyrimidinyl)-3-methylpiperazine was synthesized by utilizing 1-(5-methoxy-4-pyrimidinyl)-2-methylpiperazine in the coupling step: C₂3H₃2N₆O₃S/2.1HCl/0.4 C₂H₆O, m.p. 210°-214° C. Elemental analysis calculated: C, 50.36; H, 6.48; N, 14.81; found: C, 50.34; H, 6.47; N, 14.74.

### Example 37

### 1-[[5-[[(Ethylsulfonyl)amino[methyl]-1H-indol-3-yl]propyl-4-(5-methoxy-4-pyrimidinyl)-3-methylpiperazine

In a similar manner to Example 321-[[5-[[(ethylsulfonyl)amino]methyl]-1H-indol-3-yl]propyl]-4-(5-methoxy-4-pyrimidinyl)-3-methylpiperazine was synthesized by utilizing 1-(5-methoxy-4-pyrimidinyl)-2-methylpiperazine in the coupling step and ethanesulfonyl chloride in the sulfonylation step: C₂4H₃4N₆O₃S/2.9HCl, m.p. 2020-210° C. Elemental analysis calculated: C, 48.66; H, 6.28; N, 14.19; found: C, 48.41; H, 6.35; N, 13.93.

### Example 38

### 4-(5-Methoxy-4-pyrimidinyl)-1-[[5-[[methyl(ethylsulfonyl)amino]-methyl]-1H-indol-3-yl]propyl]piperazine

In a similar manner to Example 32 4-(5-methoxy-4-pyrimidinyl)-1-[[5-[[methyl(ethylsulfonyl)amino]methyl]-1H-indol-3-yl]propyl]piperazine was synthesized by utilizing methyl iodide in the alkylation step and ethanesulfonyl chloride in the sulfonylation step: C₂4H₃4N₆O₃S/C₄H₆O₄, m.p. 113°-115° C. Elemental analysis calculated: C, 55.61; H, 6.67; N, 13.90; found: C, 55.43; H, 6.67; N, 13.74.

### Example 39

### 4-(5-Methoxy-4-pyrimidinyl)-1-[[5-[[phenylmethyl(ethylsulfonyl)amino]-methyl]-1H-indol-3-yl]propyl]piperazine

In a similar manner to Example 32 4-(5-methoxy-4-pyrimidinyl)-1-[[5-[[phenylmethyl(ethylsulfonyl)amino]-methyl]-1H-indol-3-yl]propyl]piperazine was synthesized by utilizing benzyl bromide in the alkylation step and ethanesulfonyl chloride in the sulfonylation step: C₃0H₃8N₆O₃S/1.2C₄H₆O₄, m.p. 73°-75° C. Elemental analysis calculated: C, 59.34; H, 6.47; N, 11.93; found: C, 59.02; H, 6.17; N, 11.89.

### Example 40

### 1-[[5-[[(Acetyl)amino]methyl]-1H-indol-3-yl]propyl]-4-(5-methoxy-4-pyrimidinyl)piperazine

In a similar manner to Example 321-[[5-[[(acetyl)amino]-methyl]-1H-indol-3-yl]propyl]-4-(5-methoxy-4-pyrimidinyl)-piperazine was synthesized by utilizing acetic anhydride for acylation rather than a sulfonyl chloride for sulfonylation: C₂3H₃0N₆O₂/2.8HCl/1.2H₂O, m.p. 169°-174° C. Elemental analysis calculated: C, 50.57; H, 6.50; N, 15.39; found: C, 50.22; H, 6.87; N, 15.25.

### Example 41

### 1-[3-(5-Cyano-1H-indol-3-yl)propyl]-4-(5-methoxy-4-pyrimidinyl piperazine

To a solution of 3-(5-cyano-1H-indol-3-yl)propyl methanesulfonate (2.35 mmol, 1.0 equiv.) in 25 mL of anhydrous acetonitrile was added 0.874 g (4.5 mmol, 1.5 equiv.) of (5-methoxy-4-pyrimidinyl)piperazine and 1.0 mL of N,N-diisopropylethylamine. The mixture was heated to reflux for thirteen hours. The solution was cooled, diluted with 100 mL of chloroform and washed once with 20 mL of 10% aqueous sodium carbonate. The organic layer was separated, dried over anhydrous sodium sulfate, filtered and concentrated. The residue was chromatographed on silica gel using 5% methanol in methylene chloride containing 0.5% concentrated aqueous ammonium hydroxide to yield 0.375 g (42%, two steps) of 1-[3-(5-cyano-1H-indol-3-yl)propyl]-4-(5-methoxy-4-pyrimidinyl)piperazine pure by ¹H NMR analysis.

### Example 42

### 1-[3-(5-Aminocarbonyl-1H-indol-3-yl)propyl]-4-(5-methoxy-4-pyrimidinyl)piperazine

To a solution of 1.19 g (3.17 mmol, 1.0 equiv.) of 1-[3-(5-cyano-1H-indol-3-yl)propyl]-4-(5-methoxy-4-pyrimidinyl)piperazine in 16 mL of ethanol was added a solution of 1.85 g (33.0 mmol, 10.4 equiv.) of potassium hydroxide in 16 mL of ethanol. The solution was heated to reflux for 16 hours. TLC. indicated the reaction was less than 50% complete. The reaction mixture was diluted with 150 mL of ethyl acetate and washed once with 25 mL of saturated aqueous sodium bicarbonate. The organic layer was separated, dried over anhydrous sodium sulfate, filtered and concentrated to yield 1.22 g of an oil. The oil was chromatographed on silica gel using 10% methanol in methylene chloride containing 1% concentrated aqueous ammonium hydroxide to yield 0.293 g (23%) of 1-[3-(5-aminocarbonyl-1H-indol-3-yl)propyl]-4-(5-methoxy-4-pyrimidinyl)piperazine. Conversion to the HCl salt gave C₂1H₂4N₆O/2.8HCl/0.7H₂O, mp. 220°-225° C. (d). Elemental anal. calcd: C, 51.35; H, 5.79; N, 17.11; found: C, 51.61; H, 6.08; N, 16.82.

### Example 43

### 3-[3-[4-(5-Methoxy-4-pyrimidinyl)-1-piperazinyl]propyl]-5-aminoindole

### 5-Nitro-3-(3-bromopropyl)indole

To a solution of triphenylphosphine (6.70 g, 0.025 mol) in 80 mL of acetonitrile was added a solution of 5-nitro-3-(3-hydroxypropyl)indole (4.30 g, 0.020 mol) in 75 mL of acetonitrile, followed by a solution of CBr₄ (9.00 g, 0.027 mol) in 25 mL of acetonitrile, at 0° C. under Ar. The mixture was stirred at room temperature for 3 h and then it was evaporated and the residue was chromatographed (SiO₂/EtOAc-hexane, 1:9 then 1:4) to give the bromo compound (4.60 g, 84%) as a solid, m.p. 92°-95° C.

### 3-[3-[-4-(5-Methoxy-4-pyrimidinyl)-1-piperazinyl]propyl]-5-nitroindole

A mixture of 5-nitro-3-(3-bromopropyl)indole (0.57 g, 2.0 mmol), 1-(5-methoxy-4-pyrimidyl)piperazine (0.47 g, 2.4 mmol), KI (0.40 g, 2.4 mmol) and diisopropylethylamine (1.75 mL, 10.0 mmol) in 20 mL of acetonitrile was refluxed under Ar for 6 h. The cooled reaction mixture was diluted with ethyl acetate and washed (H₂O, brine). The aqueous wash was back-extracted with CH₂Cl₂ and the organic phase was washed (H₂O, brine). The combined organic phases were dried (Na₂SO₄) and evaporated, and the residue was chromatographed (SiO₂/CH₂Cl₂-MeOH, 95:5) to give a solid. This material was triturated with CH₂Cl₂-hexane to give the title compound (0.55 g, 70%) as a yellow solid, m.p. 163°-166° C.

### 3-[3-[4-(5-Methoxy-4-pyrimidinyl)-1-piperazinyl]propyl]-5-aminoindole

To a solution of 3-[3-[4-(5-methoxy-4-pyrimidyl)-1-piperazinyl]propyl]-5-nitroindole (0.550 g, 1.39 mmol) in a mixture of ethanol (120 mL) and THF (40 mL) was added 10% palladium-on-charcoal (0.30 g) and the mixture was hydrogenated in a Parr shaker at 40 psi for 18 h. The mixture was then filtered through Celite and the cake was washed with additional ethanol-THF. Evaporation of the filtrate gave the essentially pure title compound (0.557 g, 100%) as a brown foam. A sample of this material (0.143 g) was treated with excess methanolic HCl and the resulting solution was diluted with acetone to give a precipitate. The precipitate was filtered and then it was crystallized from ethanol to give 0.100 g of a purplish solid, m.p. 192° C. (dec). IR (KBr) 3410, 3200, 1630, 1540 cm⁻¹; ¹H NMR (DMSO-d₆, 200 MHz) δ11.22 (br s, 1H), 10.20 (br s, 2H), 8.60 (d, 1H), 8.20 (s, 1H), 7.55 (d, J=1.6 Hz, 1H), 7.55 (d, J=1.6 Hz, 1H), 7.45 (d, J=8.6 Hz, 1H), 7.35 (d, J=2.1 Hz, 1H), 7.07 (dd, J=8.6, 1.9 Hz, 1H), 4.89-4.82 (m, 2H), 3.91 (s, 3H), 3.8-3.0 (br m, 8H), 2.76 (m, 2H), 2.12 (br m, 2H). Anal. Calcd. for C₂0H₂6N₆O.4HCl.H₂O: C, 45.29; H, 6.08; N, 15.85. Found: C, 45.32; H, 5.97; N, 15.59.

### Example 44

### 3-[3-[4-(5-Methoxy-4-pyrimidinyl)-1-piperazinyl]propyl]-5-(methyl-carbamoyl)oxyindole

To a solution of 3-[3-[4-(5-methoxy-4-pyrimidinyl)-1-piperazinyl]propyl]-5-hydroxyindole (0.170 g, 0.46 mmol) in 6 mL of CH₂Cl₂ was added methyl isocyanate (60 .mu.L, 1.0 mmol) and the mixture was stirred at room temperature for 4 days. The mixture was then evaporated and the resulting pinkish-gray foam was triturated with isopropanol-ether to give a light grey solid. This material was chromatographed (SiO₂/EtOAc-MeOH, 95:5; then 90:10) to give the title compound (0.120 g, 60%) as a light gray solid, m.p. 1200-122° C. IR (KBr) 3400, 3220, 1730 cm⁻¹; ¹H NMR (CDCl₃, 200 MHz) δ8.33 (s, 1H), 8.06 (br s, 1H), 7.88 (s, 1H), 7.34-7.26 (m, 2H), 6.99-6.91 (m, 2H), 5.00 (br m, 1H), 3.85 (s, 3H), 3.79 (t, J=4.7 Hz, 4H), 2.91 (d, J=4.8 Hz, 3H), 2.75 (t, J=7.4 Hz, 2H), 2.54 (t, J=4.8 Hz, 4H), 2.45 (t, J=7.6 Hz, 2H), 2.04-1.83 (m, 2H) Anal. Calcd. for C₂2H₂8N₆O₃0.8H₂O: C, 60.20; H, 6.80; N, 19.15. Found: C, 60.13; H, 6.40; N, 18.75.

### Example 45

### 3-[3-[4-(5-Methoxy-4-pyrimidinyl)-1-piperazinyl]propyl]-5-(cyanomethyl)oxyindole

To a suspension of NaH (60% in oil, 0.020 g, 0.5 mmol) in 10 mL of dry THF was added a suspension of 3-[3-[4-(5-methoxy-4-pyrimidyl)-1-piperazinyl]propyl]-5-hydroxyindole (0.183 g, 0.5 mmol) in 20 mL of THF, at room temperature under Ar. After 30 min gas evolution had ceased and a clear solution was obtained. To this solution was added a solution of chloroacetonitrile (35 .mu.L, 0.55 mmol) in 5 mL of dry THF, and the mixture was stirred at room temperature for 2 h and then refluxed for 1½ h. The cooled mixture was diluted with CH₂Cl₂, washed (water, brine), dried (Na₂SO₄) and evaporated to give a foam. Column chromatography (SiO₂/CH₂Cl₂-MeOH, 95:5; then CH₂Cl₂-MeOH—NH₄ OH, 95:4.5:0.5) afforded the title compound (0.150 g, 75%) as a foam. This material was treated with excess methanolic HCl and the solution was evaporated to give a glass. Trituration with MeOH-EtOH (1:9) gave the hydrochloride (0.110 g) as an off-white solid, m.p. 198°-200° C. (dec): IR (KBr) 3400, 3220, 1630 cm⁻¹; ¹H NMR (DMSO-d₆, 200 MHz) δ11.39 (br s, 1H), 10.87 (br s, 1H), 8.61 (s, 1H), 8.20 (s, 1H), 7.30 (d, J=8.8 Hz, 1H), 7.24 (d, J=2.4 Hz, 1H), 7.22 (d, J=2.0 Hz, 1H), 6.83 (dd, J=8.7, 2.4 Hz, 1H), 5.16 (s, 2H), 4.90-4.83 (m, 2H), 3.90 (s, 3H), 3.70-3.36 (m, 4H), 3.13 (br s, 4H), 2.75 (m, 2H), 2.12 (m, 2H). Anal. Calcd. for C₂2H₂6N₆O₂.2.5HCl.0.3 C₂H₆O: C, 53.07; H, 5.97; N, 16.43. Found; C, 53.26; H, 5.70; N, 16.11.

### Example 46

### 3-[3-[4-(5-Methoxy-4-pyrimidinyl)-1-piperazinyl]propyl]-5-(carboxamidomethyl)oxyindole

To a suspension of NaH (60% in oil, 0.020 g, 0.5 mmol) in 5 mL of dry THF was added a suspension of 3-[3-[4-(5-methoxy-4-pyrimidyl)-1-piperazinyl]propyl]-5-hydroxyindole (0.184 g, 0.5 mmol) in 35 mL of dry THF, and the mixture was stirred at room temperature under Ar for 30 min. To the resulting clear solution was added a solution of 2-chloroacetamide (0.047 g, 0.5 mmol) in 5 mL of THF and the mixture was kept at room temperature for 2 h and then it was refluxed for 2 h. The cooled mixture was diluted with ethyl acetate, and then it was washed (water, brine), dried (Na₂SO₄) and evaporated. The residue was chromatographed (SiO₂/THF) to give impure starting material (0.117 g). Further elution afforded the title compound (0.100 g, 47%) as a gum. This material was treated with excess methanolic HCl and the solution was then evaporated and the residue was crystallized from methanol-ether to give the hydrochloride (0.095 g) as a grayish solid, m.p. 90° C.: IR (KBr) 3400, 3250, 1680, 1630 cm⁻¹; ¹H NMR (DMSO-d₆, 200 MHz) δ 11.55 (br s, 1H), 10.76 (br s, 1H), 8.69 (s, 1H), 8.21 (s, 1H), 7.49-7.40 (br m, 2H), 7.25 (d, J=8.6 Hz, 1H), 7.16 (d, J=2.1 Hz, 1H), 7.05 (d, J=2.2 Hz, 1H), 6.81 (dd, J=8.7, 2.3 Hz, 1H), 4.99-4.92 (br m, 2H), 4.41 (s, 2H), 3.91 (s, 3H), 4.3-3.5 (m, 4H), 3.11 (br s, 4H), 2.72 (m, 2H), 2.10 (br s, 2H). Anal. Calcd. for C₂2H₂8N₆O₃.3HCl.H₂O: C, 47.87; H, 6.03; N, 15.23. Found: C, 48.24; H, 5.89; N, 14.83.

### Example 47

1-[3-[5-acetyl-1H-indol-3-yl]propyl]-4-(5-methoxy-4-pyrimidin yl)piperazine hydrochloride was prepared in the usual manner from 1-[5-acetyl-1H-indol-3-yl]-3-propanol. m.p. 215°-217° (d). Analysis calcd. for C₂2H₂7N₅O₂/2.7HCl: C, 53.72; H, 6.09; N, 14.24. found: C, 53.52; H, 6.23; N, 14.28.

### Example 48

### 4-(5-Methoxy-4-pyrimidinyl)-1-[3-[5-[2-(methylsulfonyl)methyl]-1H-indol-3-yl]propyl]piperazine

m.p. 190°-195° C. Analysis calcd. for C₂2H₂9N₅O₃S.3.0HCl: C, 47.79; H, 5.84; N, 12.67;

Found: C, 47.58; H, 5.96; N, 12.32.

### Example 49

### 4-(5-Methoxy-4-pyrimidinyl)-1-[3-[5-[2-(methylsulfonyl)methyl]-1H-indol-3-yl]propyl]-3-methylpiperazine

m.p. 208°-210(d)° C. Analysis calcd. for C₂3H₃2N₅O₃S.2.5HCl: C, 50.25; H, 6.33; N, 12.47; Found: C, 50.17; H, 6.04; N, 12.58.

### Example 50

### 4-(5-Methoxy-4-pyrimidinyl)-1-[3-[5-[2-(methylsulfonyl)ethyl]-1H-indol-3-yl ]propyl]piperazine

m.p 153°-154° C. Analysis calcd. for C₂3H₃1N₅O₃S: C, 60.37; H, 6.83; N, 15.30; Found: C, 60.24; H, 6.79; N, 15.42.

### Example 51

### 4-(5-Methoxy-4-pyrimidinyl)-1-[3-[5-[2-(methylsulfonyl)ethyl]-1H-indol-3-yl]propyl]-3-methylpiperazine

m.p. 220°-223° C. Analysis calcd. for C₂4H₃3N₅O₃S.2.5HCl: C, 51.22; H, 6.36; N, 12.44; Found: C, 51.20; H, 6.26; N, 12.36.

### Example 52

### 4-(5-Ethoxy-4-pyrimidinyl)-1-[3-(1H-indol-3-yl)propyl]piperazine hydrochloride hydrate

A mixture of 3-(1H-indole-3-yl)propyl 4-methylbenzenesulfonate (0.87 g, 2.65 mmol), 1-(5-ethoxy-4-pyrimidinyl)piperazine (1.10 g, 5.29 mmol), micropulverized K₂CO₃ (0.73 g, 5.29 mmol), and tetrabutylammonium hydrogen sulfate (0.04 g, 0.13 mmol) in CH₃ CN (15 mL) was heated at reflux for 2.5 h under nitrogen atmosphere. The reaction was allowed to cool to 23° C. and stand for 16 h. The reaction was concentrated under reduced pressure and the residue was dissolved in CH₂Cl₂ and extracted with water. The organic phase was dried with anhydrous K₂CO₃, filtered, and concentrated under reduced pressure. Silica gel chromatography (CH₂Cl₂/MeOH; 98:2) of the residue afforded the free base (0.70 g, 72%) which was treated with ethanolic HC.1 to yield I (0.85 g, 99%; mp >230° C.) after crystallization from EtOH/MeOH.

Anal. Calcd. for C₂1H₂7N₅O.2HCl.0.7H₂O:

C, 55.93; H, 6.80; N, 15.53; H₂O, 2.80.

Found: C, 55.67; H, 6.66; N, 15.40; H₂O, 2.50.

### Example 53

### 1-[3-(1H-indol-3-yl)butyl]-4-(5-methoxy-4-pyrimidinyl)piperazine

To a mixture of 1-(5-methoxy-4-pyrimidyl)piperazine (1.55 g, 8.0 mmol), triethylamine hydrochloride (1.10 g, 8.0 mmol) and NaCNBH₃ (1.76 g, 28 mmol) in 20 mL of dry THF was added a solution of 3-(3-oxobutyl)indole, J. Szmuskovicz, et al., J. Am. Chem. Soc. 79, 2819 (1957), (0.75 g, 4.0 mmol) in 5 mL of THF and the mixture was stirred at room temperature under Ar for 20 h. The reaction mixture was then poured into 10% saturated NaHCO₃ and extracted with ethyl acetate (.times.2). The organic extract was washed with H₂O (.times.2) and then with 25 mL of 0.1N HCl. The resulting organic solution was extracted with 1N HCl (.times.2) and the aqueous phase was washed with CH₂Cl₂ (.times.2) and then it was cooled at 0° C. and basified with 50% aqueous NaOH. This gave a gummy precipitate which was extracted into ethyl acetate (.times.4) and the combined organic extract was then washed (brine), dried (Na₂SO₄) and evaporated to give a gum. Flash chromatography (SiO₂/acetonitrile-methanol; 95:5 then 80:20) of this material gave the pure product (968 mg, 66%) as a white foam.

The foam was taken up in CH₂Cl₂ and treated with excess ethanolic HCl. The solution was evaporated and the residue was again taken up in excess ethanolic HCl. Evaporation of the solution gave a light brown foam which was crystallized from hot methanol-acetone to give the hydrochloride (916 mg) as a white powder: m.p. 169°-72° C. (dec); IR (KBr) 3400, 1632, 1550, 1275 cm⁻¹; ¹H nmr (200 MHz, CDCl₃) δ11.68 (br s, 1H), 10.89 (s, 1H), 8.66 (s, 1H), 8.20 (s, 1H), 7.56 (d, J=7.4 Hz, 1H), 7.33 (d, J=7.7 Hz, 1H), 7.21 (d, J=2.3 Hz), 7.10-6.93 (m, 2H), 5.03-4.96 (m, 2H), 3.91 (s, 3H), 3.85-3.75 (m, 2H), 3.54-3.25 (m, 5H), 2.88-2.60 (m, 2H), 2.51-2.35 (m, 1H), 1.88-1.81 (m, 1H), 1.38 (d, J=6.5 Hz, 3H).

Anal. Calcd. for C₂1H₂7N₅O.2HCl.1.4H₂O:

C, 54.50; H, 6.91; N, 15.11.

Found: C, 54.39; H, 6.93; N, 15.37.

### Example 54

### 3-[3-[4-(5-Methoxy-4-pyrimidinyl)-1-piperazinyl]pentyl]indole

A mixture of 3-(3-bromopentyl)indole (1.33 g, 5.0 mmol), 1-(5-methoxy-4-pyrimidyl)piperazine (2.00 g, 10 mmol) finely pulverized K₂CO₃ (0.70 g, 5.1 mmol) and finely pulverized KI (0.90 g, 5.1 mmol) in 20 mL of acetonitrile was heated to reflux under Ar for 5 h. The cooled reaction mixture was diluted with EtOAc, washed (H₂O, brine), dried (NO₂SO₄) and evaporated to give a gum. Chromatographs of this material (S10₂/EtOAc then 10% MeOH-EtOAc) affords the product (0.65 g, 34%) as a gum. This gum was taken up in ether and then methanolic HCl was added. The resulting solid was filtered, washed with Et₂O and dried in vacuo to give an off-white solid (0.50 g), mp 145° C. (dec); IR (KBr) 3400, 1633, 1550 cm⁻¹;

Anal. Calcd. for C₂2H₂9N₅O.3HCl.0.75H₂O:

C, 52.59; H, 6.72; N, 13.94.

Found: C, 52.70; H, 6.34; N, 13.91.

### Example 55

### 1-[3-(5-Hydroxy-1H-indol-3-yl)propyl]-4-(5-methoxy-4-pyrimidinyl)piperazine

A mixture of 1-[3-(5-benzyloxy-1H-indol-3-yl)propyl]-4-(5-methoxy-4-pyrimidinyl)piperazine (Ex. 71; 1.40 g, 3.06 mmol) and 10% Pd(OH₂)/C (0.85 g) in 25 mL of ethanol was hydrogenated at 30-40 psi in a Parr shaker for 3.5 h. The mixture was filtered and then fresh catalyst (0.75 g) was added to the filtrate and hydrogenation was resumed at ca 45 psi for 22 h. The resulting mixture was filtered through Celite and the filtrate was evaporated to give a foam (0.995 g, 88%). The foam was taken up in methanolic HCl, whereupon a white solid precipitated. The solid was filtered, washed with cold methanol and then ether, and dried in vacuo to give the hydrochloride as a slightly pinkish solid (0.59 g), mp 215° C.; IR (KBr) 3320 (Br) 1630, 1550 cm⁻¹; 7.07 (s, 1H), 6.80 (d, J=2.1 Hz, 1H), 6.59 (dd, J=2.2, 8.6 Hz, 1H), 4.91-4.84 (m, 2H), 3.90 (s, 3H), 3.8-3.4 (m, 7H), 3.2-3.0 (m, 4H), 2.69-2.62 (m, 2H), 2.2-2.0 (m, 2H).

Anal. Calcd. for C₂0H₂5N₅O₂.2.25HCl:

C, 53.44; H, 6.11; N, 15.58.

Found: C, 53.30; H, 5.90; N, 15.40.

### EXAMPLE 56

### N-Butyl-3-[3-[4-(5-methoxy-4-pyrimidinyl)piperazin-1-yl]prop-1-yl]-1H-indole-5-carboxamide hydrochloride

Sodium metal (0.84 g, 36.67 mmol) was dissolved in 15 mL of anhydrous methanol and n-butylamine (2.68 g, 36.67 mmol) at 0+C. was added. A solution of methyl 3-[3-[4-[(5-methoxy-4-pyrimidinyl)piperazin-1-yl]prop-1-yl]-1H-indole-5-carboxylate (1.5 g, 3.67 mmol) in 5 mL of methanol was added. The reaction was heated at reflux for 24 hours. The reaction was cooled and quenched with 5 mL of water. The methanol was removed in vacuo. The residue was extracted with three 30 mL portions of ethyl acetate. The combined organic layers were dried over anhydrous sodium sulfate, filtered and concentrated in vacuo. Silica gel chromatography (94:6:0.5 CH₂Cl₂-MeOH—NH₄₀H) of the concentrate yielded N-butyl-3-[3-[4-(5-methoxy-4-pyrimidinyl)piperazin-1-yl]prop-1-yl]-1H-indole-5-carboxamide (0.59 g, 36%). The hydrochloride salt was prepared from 0.59 g of N-butyl-3-[3-[4-(5-methoxy-4-pyrimidinyl)piperazin-1-yl]prop-1-yl]-1H-indole-5-carboxamide to yield the title compound (0.23 g, 36%): mp 180°-185° C. Anal. Calcd for C₂5H₃4N₆O₂.2.0HCl: C, 57.36; H, 6.93; N, 16.05. Found: C, 57.01; H, 6.95; N, 15.98.

### Example 57

### 1-[3-[5-[(Trifluoromethyl)carbonyl]amino-1-H-indol-3-yl]propyl]-4-(5-methoxy-4-pyrimidinyl)piperazine hydrochloride

To a solution of 1-[3-[5-amino-1-H-indol-3-yl]propyl]-4-(5-methoxy-4-pyrimidinyl)piperazine (0.5 g, 1.36 mmol) in methylene chloride (8 mL) at 0° C. was added trifluoroacetic anhydride (0.34 g, 1.63 mmol). The reaction mixture was allowed to warm to room temperature over 2 h. The solution was diluted with water, and the organic layer was dried (MgSO₄), filtered and concentrated in vacuo. Silica gel column chromatography (95:5 ethyl acetate-methanol) of the concentrate gave 1-[3-[5-[(trifluoromethyl)carbonyl]amino-1-H-indol-3-yl]propyl]-4-(5-methoxy-4-pyrimidinyl)piperazine (0.2 g, 32%). The free base (0.2 g, 0.43 mmol) was dissolved in a minimum volume of ethanol, and was converted to its hydrochloride salt with 2 mL of 3 N HCl in EtOH. The volatile components were removed in vacuo to give the title compound (0.19 g, 0.28 mmol, 67%), mp 295°-300° C. Anal. Calcd for C₂2H₂5F₃N₆O₂.5.5HCl: C, 39.86; H, 4.64; N, 12.68. Found: C, 40.05; H, 5.03; N, 12.33.

### Example 58

### 1-[3-[5-[[(4-Methylphenyl)sulfonyl]amino]-1-H-indol-3-yl]propyl]-4-(5-methoxy-4-pyrimidinyl)piperazine oxalate

p-Toluenesulfonyl chloride (0.30 g, 1.56 mmol) was dissolved in 2 mL of THF, and the solution was cooled to 0° C. A mixture of 1-[3-[5-amino-1-H-indol-3-yl]propyl]-4-(5-methoxy-4-pyrimidinyl)piperazine (0.5 g, 1.42 mmol), and triethylamine (0.16 g, 1.56 mmol) in 3 mL of THF was added to the p-toluenesulfonyl chloride solution. The solution was stirred at 0° C. for 30 min, warmed to 23° C. (1 h), and concentrated in vacuo. Silica gel column chromatography (95:5:0.5 CH₂Cl₂-MeOH-30% NH₄₀H) of the concentrate gave 1-[3-[5-[[(4-methylphenyl) sulfonyl]amino]-1-H-indol-3-yl]propyl]-4-(5-methoxy-4-pyrimidinyl)piperazine (0.42 g, 0.8 mmol, 57%). The free base (0.42 g, 0.80 mmol) was dissolved in a minimum volume of acetonitrile. A concentrated solution of oxalic acid (0.072 g, 0.8 mmol) in CH₃CN was added with stirring. The precipitate was separated by filtration, washed sparingly with CH₃CN and dried at 70° C. in vacuo overnight to give the title compound (0.40 g, 0.66 mmol, 82%): mp 125°-127° C. Anal. Calcd for C₂7H₃2N₆O₃S₁.1.0C₂H₂O₄: C, 55.40; H, 5.77; N, 13.36. Found: C, 55.29; H, 5.41; N, 13.15.

### Example 59

### 1-[3-[5-[2-Pyrrolidinon-1-yl]-1H-indol-3-yl]propyl]-4-(5-methoxy-4-pyrimidinyl)piperazine oxalate

To a mixture of 1-[3-[5-amino-1-H-indol-3-yl]propyl]-4-(5-methoxy-4-pyrimidinyl)piperazine (2.3 g, 6.3 mmol), and sodium carbonate (0.67 g, 6.3 mmol) in 25 mL of acetone, cooled to 0° C., was added dropwise 4-chlorobutyryl chloride (0.89 g, 6.3 mmol). Additional acetone (10 mL) was added and the suspension was stirred for 19 h at 23° C. The insoluble material was separated by filtration and the acetone was removed in vacuo. The crude material was dissolved in EtOAc, and the solution was washed with water, dried (brine, MgSO₄), filtered, and concentrated in vacuo to give crude 1-[3-[5-[4-chlorobutyrylamino]-1-H-indol-3-yl]propyl]-4-(5-methoxy-4-pyrimidinyl)piperazine (1.98 g, 4.2 mmol, 67%) as a gum. Sodium ethoxide (0.67 mL, 2.1 mmol, 21% in ethanol) was added dropwise to a solution of crude 1-[3-[5-[4-chlorobutyrylamino]-1-H-indol-3-yl]propyl]-4-(5-methoxy-4-pyrimidinyl)piperazine (1.0 g, 2.1 mmol) in ethanol (5 mL). After the addition, the reaction mixture was heated at 78° C. for 90 min and then cooled to room temperature. The insoluble solid was separated and the filtrate was concentrated in vacuo. Silica gel column chromatography (95:5:0.5 CH₂Cl₂-MeOH-30% NH₄₀H) afforded 1-[3-[5-[2-pyrrolidinone-1-yl]-1-yl]-1-H-indol-3-yl]propyl]-4-(5-methoxy-4-pyrimidinyl)piperazine (0.33 g, 0.76 mmol, 36%). The free base (0.25 g, 0.58 mmol) was dissolved in a minimum volume of acetonitrile. A concentrated solution of oxalic acid (0.052 g, 0.58 mmol) in CH₃CN was added with stirring. The precipitate was separated by filtration, washed sparingly with CH₃ CN, and dried at 70° C. in vacuo overnight to give the title compound (0.17 g, 56%): mp 100°-101° C. Anal. Calcd for C₂4H₃0N₆O₂.1.0 C₂H₂O₄.1.15H₂O: C, 57.26; H, 6.34; N, 15.41. Found: C, 56.90; H, 5.96; N, 15.51.

By appropriate modification of the foregoing synthetic examples, additional Formula 1 compounds may be obtained. Selected additional example compounds are set forth in Table 1.

TABLE 1
Additional Products of Formula I

| | |
|---|---|
| | I |
| 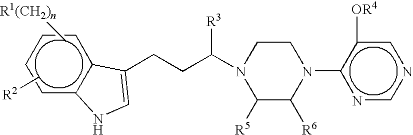 | |

| | | | | | | | | MP ° C. of HCl salt |
|---|---|---|---|---|---|---|---|---|
| Example | | | | | | | | (unless otherwise |
| No. | R¹ | n | R² | R³ | R⁴ | R⁵ | R⁶ | specified) |
| 60 | 5-F | 0 | H | H | Me | H | H | 119-122 (base) |
| 61 | H | 0 | H | H | Me | H | H | 221-224 |
| 62 | H | 0 | H | H | Me | H | Me | 185 (dec) |
| 63 | 5-MeSO₂NH | 0 | H | H | Me | H | H | 150-152 |
| 64 | 5-MeSO₂NMe | 0 | H | H | Me | H | Me | 205-206 |
| 65 | 5-MeSO₂NMe | 0 | H | H | Me | H | H | 227-230 |
| 66 | 5-MeSO₂NMe | 0 | H | H | Me | H | Me | 215-218 |
| 67 | 5-EtSO₂NH | 0 | H | H | Me | H | H | 140-145 |
| 68 | 5-EtSO₂NMe | 0 | H | H | Me | H | H | 223-225 |
| 69 | 5-EtSO₂NMe | 0 | H | H | Me | H | Me | 205-207 |
| 70 | 5-EtSO₂NH | 0 | H | H | Me | H | Me | 212-214 |
| 71 | 5-EtO | 0 | H | H | Me | H | H | 112-114 (base) |
| 72 | 5-PhCH₂O | 0 | H | H | Me | H | H | 180 (dec) |
| 73 | 5-MeO | 0 | H | H | Me | H | H | 130-150 (dec) |
| 74 | 5-F | 0 | 6-F | H | Me | H | H | 102-104 (base) |
| 75 | 4-F | 0 | 7-F | H | Me | H | H | 160-165 (base) |
| 76 | 5-F | 0 | 7-F | H | Me | H | H | >250 (dec) |
| 77 | 5-H₂NSO₂ | 1 | H | H | Me | H | H | 125-127 (succinate) |
| 78 | 5-H₂NCO | 1 | H | H | Me | H | H | 170 (dec) |
| 79 | 5-CN | 1 | H | H | Me | H | H | 163-165 (fumarate) |
| 80 | 5-PhCH₂NHCO | 1 | H | H | Me | H | H | 112-115 |
| 81 | 5-EtO₂C | 0 | H | H | Me | H | H | 215-216 |
| 82 | 5-MeO₂C | 0 | H | H | Me | H | H | — |
| 83 | 5-MeNHCO | 0 | H | H | Me | H | H | >215 |
| 84 | 5-EtNHCO | 0 | H | H | Me | H | H | >215 |
| 85 | 5-PhCH₂CH₂NHCO | 0 | H | H | Me | H | H | 155-165 |
| 86 | 5-PhCH₂NHCO | 0 | H | H | Me | H | H | 190-195 |
| 87 | 5-CHONH | 0 | H | H | Me | H | H | 118-119 (oxalate) |
| 88 | 5-CH₃CONH | 0 | H | H | Me | H | H | 135-137 (oxalate) |
| 89 | 5-F | 0 | H | H | Me | Me | H | 215-218 |
| 90 | 5-F | 0 | 6-CO₂Me | H | Me | H | H | 225-230 darkens |
| 91 | 5-F | 0 | 6-MeO | H | Me | H | H | 236-238 |

TABLE 2
5-HT_{1D} Binding Site Affinities For
Representative Formula I Compounds

| | 5-HT_{1D} Binding |
|---|---|
| Example No. | IC₅₀ (nM) |
| 52 | 2.0 |
| 53 | 5.0 |
| 54 | 20.5 |
| 55 | 0.8 |
| 60 | 3.0 |
| 61 | 2.9 |
| 62 | 4.2 |
| 63 | 5.2 |
| 64 | 9.6 |
| 65 | 4.9 |
| 66 | 12.0 |
| 67 | 13.7 |
| 68 | 12.9 |
| 69 | 19.1 |
| 70 | 17.1 |
| 71 | 2.2 |
| 72 | 14.3 |
| 73 | 1.1 |
| 74 | 29.0 |
| 75 | 5.1 |
| 76 | 8.1 |

A preferred embodiment of the present products of Formula I is represented above in Tables 1 and 2 as Example 60. This example has the following formula:

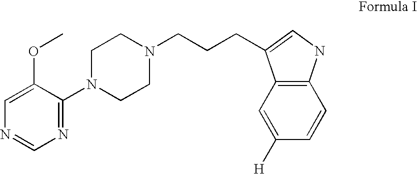

This preferred embodiment, 3-(3-(4-(5-methoxy-4-pyrimidinyl)-1-piperazinyl)propyl)-5-fluoro-1H-indole hydrochloride, or 5-Fluoro-3-[3-[4-(5-methoxypyrimidin-4-yl)piperazin-1-yl]propyl]-1H-indole, is known in the literature as BMS-181101. This embodiment of Formula I, and its metabolites, are useful for the treatment of anxiety, depression, and sexual dysfunction. The process for making this embodiment is described in Anderson, N. G.; et al.; Process development of 5-fluoro-3[3-[4-(5-methoxy-4-pyrimidinyl)-1-piperazinyl]propyl]-1H-indole dihydrochloride, Org. Process Res. Dev. 1997, 1, 4, 300, herein incorporated by reference in its entirety. An example scheme for manufacture of 5-Fluoro-3-[3-[4-(5-methoxypyrimidin-4-yl)piperazin-1-yl]propyl]-1H-indole is shown as Scheme 5 below:

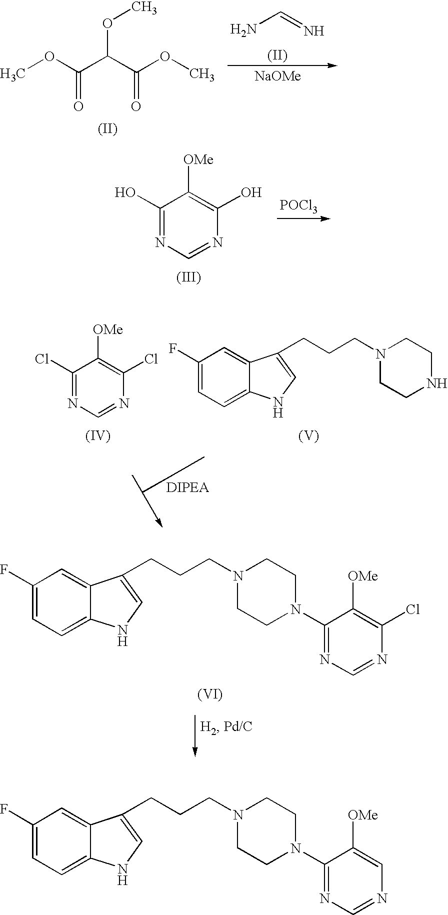

Obviously, numerous modifications and variations of the present invention are possible in light of the above teachings. It is therefore to be understood that within the scope of the appended claims, the invention may be practiced otherwise than as specifically described herein.

1. A method of treating or preventing anxiety, or symptoms thereof, in a patient in need thereof comprising administering a therapeutically effective amount of 3-(3-(4-(5-methoxy-4-pyrimidinyl)-1-piperazinyl)propyl)-5-fluoro-1H-indole hydrochloride or a metabolite thereof, or a pharmaceutically acceptable salt thereof.

2. The method of claim 1, wherein the 3-(3-(4-(5-methoxy-4-pyrimidinyl)-1-piperazinyl)propyl)-5-fluoro-1H-indole hydrochloride or metabolite thereof is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

3. The method of claim 1, wherein the 3-(3-(4-(5-methoxy-4-pyrimidinyl)-1-piperazinyl)propyl)-5-fluoro-1H-indole hydrochloride or metabolite thereof is administered with a pharmaceutically acceptable carrier.

4. The method of claim 1, wherein said administering is selected from the group consisting of oral, rectal, nasal, parenteral, intracisternal, intravaginal, intraperitoneal, sublingual, topical, and bucal.

5. The method of claim 4, wherein said administering is oral or parenteral.

6. The method of claim 1, wherein the patient in need thereof also suffers from one or more disorders selected from the group consisting of depression, obesity, drug abuse/addiction, alcohol abuse, sleep disorders, TIC disorder, sexual dysfunction, and behavioral/cognitive symptoms of Alzheimer's disease.

7. A method of treating or preventing depression, or symptoms thereof, in a patient in need thereof comprising administering a therapeutically effective amount of 3-(3-(4-(5-methoxy-4-pyrimidinyl)-1-piperazinyl)propyl)-5-fluoro-1H-indole hydrochloride or a metabolite thereof, or a pharmaceutically acceptable salt thereof.

8. The method of claim 7, wherein the 3-(3-(4-(5-methoxy-4-pyrimidinyl)-1-piperazinyl)propyl)-5-fluoro-1H-indole hydrochloride or metabolite thereof is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

9. The method of claim 7, wherein the 3-(3-(4-(5-methoxy-4-pyrimidinyl)-1-piperazinyl)propyl)-5-fluoro-1H-indole hydrochloride or metabolite thereof is administered with a pharmaceutically acceptable carrier.

10. The method of claim 7, wherein said administering is selected from the group consisting of oral, rectal, nasal, parenteral, intracisternal, intravaginal, intraperitoneal, sublingual, topical, and bucal.

11. The method of claim 10, wherein said administering is oral or parenteral.

12. The method of claim 7, wherein the patient in need thereof also suffers from one or more disorders selected from the group consisting of anxiety, obesity, drug abuse/addiction, alcohol abuse, sleep disorders, TIC disorder, sexual dysfunction, and behavioral/cognitive symptoms of Alzheimer's disease.

13. A method of treating or preventing sexual dysfunction, or symptoms thereof, in a patient in need thereof comprising administering a therapeutically effective amount of 3-(3-(4-(5-methoxy-4-pyrimidinyl)-1-piperazinyl)propyl)-5-fluoro-1H-indole hydrochloride or a metabolite thereof, or a pharmaceutically acceptable salt thereof.

14. The method of claim 13, wherein the 3-(3-(4-(5-methoxy-4-pyrimidinyl)-1-piperazinyl)propyl)-5-fluoro-1H-indole hydrochloride or metabolite thereof is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

15. The method of claim 13, wherein the 3-(3-(4-(5-methoxy-4-pyrimidinyl)-1-piperazinyl)propyl)-5-fluoro-1H-indole hydrochloride or metabolite thereof is administered with a pharmaceutically acceptable carrier.

16. The method of claim 13, wherein said administering is selected from the group consisting of oral, rectal, nasal, parenteral, intracisternal, intravaginal, intraperitoneal, sublingual, topical, and bucal.

17. The method of claim 16, wherein said administering is oral or parenteral.

18. The method of claim 13, wherein the patient in need thereof also suffers from one or more disorders selected from the group consisting of depression, anxiety, obesity, drug abuse/addiction, alcohol abuse, sleep disorders, TIC disorder, and behavioral/cognitive symptoms of Alzheimer's disease.
